(19) United States
(12) Patent Application Publication
(10) Pub. No.: US 2008/0269522 A1
(43) Pub. Date: Oct. 30, 2008

(54) PROCESS FOR OPERATING AN EXOTHERMIC HETEROGENEOUSLY CATALYZED PARTIAL GAS PHASE OXIDATION OF AN ORGANIC STARTING COMPOUND TO AN ORGANIC TARGET COMPOUND

(75) Inventors: Jochen PETZOLDT, Weisenheim am Berg (DE); Martin Dieterle, Jersey City, NJ (US); Klaus Joachim Muller-Engel, Stutensee (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/042,671

(22) Filed: Mar. 5, 2008

Related U.S. Application Data
(63) Continuation of application No. PCT/EP07/62186, filed on Nov. 12, 2007.
(60) Provisional application No. 60/865,929, filed on Nov. 15, 2006.
(60) Provisional application No. 60/868,631, filed on Dec. 5, 2006.

(30) Foreign Application Priority Data
Nov. 15, 2006 (DE) 102006054214.2
Dec. 5, 2006 (DE) 102006057631.4

(51) Int. Cl. C07C 51/16

(52) U.S. Cl. 562/545

(57) ABSTRACT

A process for operating an exothermic heterogeneously catalyzed partial gas phase oxidation of an organic starting compound in a tube bundle reactor cooled with a fluid heat carrier, in which an increase in the loading of the fixed catalyst bed disposed in the reaction tubes with the organic starting compound is preceded by an increase in the inlet temperature of the fluid heat carrier into the space surrounding the reaction tubes.

The present invention relates to a process for operating an exothermic heterogeneously catalyzed partial gas phase oxidation of an organic starting compound to an organic target compound in different operating states I and II, in which a reaction gas input mixture comprising the organic starting compound, molecular oxygen and at least one inert diluent gas is passed through a fixed catalyst bed disposed in the tubes of a tube bundle reactor to obtain a product gas mixture comprising the organic target compound, and the reaction temperature in the fixed catalyst bed disposed in the tubes is adjusted by conducting at least one fluid heat carrier into the space surrounding the tubes of the tube bundle reactor which comprise the fixed catalyst bed with an entrance temperature Tⁱⁿ and out of it again with an exit temperature T^{out}>Tⁱⁿ, and the fixed catalyst bed is loaded in the operating state I at an entrance temperature T_{I}ⁱⁿ with a load L^{I} of the organic starting compound and, in the operating state II, at an entrance temperature T_{II}ⁱⁿ in with a load L^{II} of the organic starting compound, with the proviso that L^{II}>L^{I} and T_{II}ⁱⁿ>T_{I}ⁱⁿ.

A full oxidation of an organic compound with molecular oxygen is understood here to mean that the organic compound is converted with the reactive action of molecular oxygen in such a way that all of the carbon present in the organic compound is converted to oxides of carbon, and all of the hydrogen present in the organic compound to oxides of hydrogen. All different reactions of an organic compound with the reactive action of molecular oxygen are summarized here as partial oxidation of an organic compound.

In particular, partial oxidation shall be understood here to mean those conversions of organic compounds under the reactive action of molecular oxygen in which the organic compound to be oxidized partially, on completion of conversion, comprises at least one more oxygen atom in chemically bound form than before the partial oxidation was carried out.

In this document, a diluent gas behaving essentially inertly under the conditions of a heterogeneously catalyzed gas phase partial oxidation is understood to mean those diluent gases whose constituents, under the conditions of the heterogeneously catalyzed gas phase partial oxidation, each constituent viewed alone, remain chemically unchanged to an extent of more than 95 mol %, preferably to an extent of more than 99 mol %.

In this document, the load of a fixed catalyst bed catalyzing a reaction step with reaction gas mixture is understood to mean the amount of reaction gas mixture in standard liters (=I(STP); the volume in liters which would be taken up by the corresponding amount of reaction gas mixture under standard conditions, i.e. at 0° C. and 1 bar) which is fed to the fixed catalyst bed per hour based on the volume of its bed, i.e. on its bed volume (pure inert material sections are not taken into account) (→unit=I(STP)/I·h). The load may also be based only on one constituent of the reaction gas mixture (for example on the organic starting compound of the partial oxidation). In that case, it is the amount of this constituent (for example the organic starting compound of the partial oxidation) which is fed per hour to the fixed catalyst bed, based on the volume of its bed (in the case of the organic starting compound of the partial oxidation, the load in this document is also referred to as L).

It is common knowledge that partial and heterogeneously catalyzed oxidation of a very wide variety of organic precursor compounds with molecular oxygen in the gas phase allows numerous commodity chemicals (organic target compounds, target products) to be obtained. Examples include (all of them are useful for the process according to the invention) the conversion of propylene to acrolein and/or acrylic acid (cf., for example, DE-A 23 51 151), the conversion of tert-butanol, isobutene, isobutane, isobutyraldehyde or the methyl ether of tert-butanol to methacrolein and/or methacrylic acid (cf., for example, DE-A 25 26 238, EP-A 92097, EP-A 58927, DE-A 41 32 263, DE-A 41 32 684 and DE-A 40 22 212), the conversion of acrolein to acrylic acid, the conversion of methacrolein to methacrylic acid (cf., for example, DE-A 25 26 238), the conversion of o-xylene, p-xylene and/or naphthalene to phthalic anhydride (cf., for example, EP-A 522 871) or the corresponding acids, and the conversion of butadiene to maleic anhydride (cf., for example, DE-A 21 06 796 and DE-A 16 24 921), the conversion of n-butane to maleic anhydride (cf., for example, GB-A 14 64 198 and GB-A 12 91 354), the conversion of indanes to, for example, anthraquinone (cf., for example, DE-A 20 25 430), the conversion of ethylene to ethlyene oxide or of propylene to propylene oxide (cf., for example, DE-B 12 54 137, DE-A 21 59 346, EP-A 372 972, WO 89/07101, DE-A 43 11 608 and Beyer, Lehrbuch der organischen Chemie [Textbook of organic chemistry], 17th edition (1973), Hirzel Verlag, Stuttgart, page 261), the conversion of propylene and/or acrolein to acrylonitrile (cf., for example, DE-A 23 51 151), the conversion of isobutene and/or methacrolein to methacrylonitrile (i.e. the term “partial oxidation” in this document shall also encompass partial ammoxidation, i.e. a partial oxidation in the presence of ammonia), the oxidative dehydrogenation of hydrocarbons (cf., for example, DE-A 23 51 151), the conversion of propane to acrylonitrile, or to acrolein and/or to acrylic acid (cf., for example, DE-A 10 13 1297, EP-A 1090 684, EP-A 608 838, DE-A 10 04 6672, EP-A 529 853, WO 01/96270 and DE-A 10 02 8582), the conversion of isobutane to methacrolein and/or methacrylic acid, and also the reactions of ethane to give acetic acid, of ethylene to give ethylene oxide, of benzene to give phenol, and of 1-butene or 2-butene to give the corresponding butanediols, etc.

The fixed catalyst bed has the task of causing the desired gas phase partial oxidation to proceed preferentially over the full oxidation. The chemical reaction is effected when the reaction gas mixture flows through the fixed bed during the residence time of the reaction gas mixture therein.

The solid state catalysts to be used are frequently oxide compositions or noble metals (e.g. Ag). The catalytically active oxide composition may, as well as oxygen, comprise only one other element or more than one other element (multielement oxide compositions). The catalytically active oxide compositions used are particularly frequently those which comprise more than one metallic, especially transition metal, element. In this case, reference is made to multimetal oxide compositions. Typically, these are not simple physical mixtures of oxides of the elemental constituents but rather heterogeneous mixtures of complex poly compounds of these elements.

In practice, the aforementioned catalytically active solid compositions are generally used shaped to a wide variety of different geometries (rings, solid cylinders, spheres, etc.). The shaping (to give the shaped body) can be effected in such a way that the catalytically active composition is shaped as such (for example in extruders or tableting apparatus), so as to result in a so-called unsupported catalyst, or by applying the active composition to a preshaped support (cf., for example, WO 2004/009525 and WO 2005/113127).

Examples of catalysts which are suitable for inventive heterogeneously catalyzed gas phase partial oxidations in the fixed catalyst bed of at least one organic starting compound can be found, for example, in DE-A 10046957, in EP-A 1097745, in DE-A 4431957, in DE-A 10046928, in DE-A 19910506, in DE-A 19622331, in DE-A 10121592, in EP-A 700714, in DE-A 19910508, in EP-A 415347, in EP-A 471853 and in EP-A 700893.

Usually, heterogeneously catalyzed gas phase partial oxidations, especially those mentioned at the outset of this document, are performed at elevated reaction temperatures (generally a few hundred ° C., typically from 100 to 600° C.).

The working pressure (absolute pressure) in heterogeneously catalyzed gas phase partial oxidations may be below 1 atm, at 1 atm or above 1 atm. In general, it is from 1 to 10 atm, usually from 1 to 3 atm.

Owing to the normally markedly exothermic character of heterogeneously catalyzed gas phase partial oxidations of organic compounds with molecular oxygen, the reactants are typically diluted with a gas which is essentially inert under the conditions of the catalytic partial oxidation in the gas phase and is capable of absorbing heat of reaction released with its heat capacity.

One of the most frequently used inert diluent gases is molecular nitrogen which is used automatically whenever the oxygen source used for the heterogeneously catalyzed gas phase partial oxidation is air.

Owing to its general availability, another inert diluent gas which is used in many cases is steam. In addition, both nitrogen and steam are advantageously uncombustible inert diluent gases.

In many cases, cycle gas is also used as an inert diluent gas (cf., for example, EP-A 1180508), or dilution is effected exclusively with cycle gas. Cycle gas refers to the residual gas which remains after a one-stage or multistage (in the multistage heterogeneously catalyzed gas phase partial oxidation of organic compounds, the gas phase partial oxidation, in contrast to the one-stage heterogeneously catalyzed gas phase partial oxidation, is carried out not in one reactor, but rather in at least two reactors connected in series (which can merge into one another seamlessly in a common casing (for example in the case of a single reactor)) and form separate reaction zones therein, in which case oxidant can be supplemented if appropriate between successive reactors; multiple stages are employed especially when the partial oxidation proceeds in successive steps; in these cases, it is frequently appropriate to optimize both the catalyst and the other reaction conditions to the particular reaction step and to carry out the reaction step in a dedicated reactor (or in a separate (dedicated) reaction zone of a reactor), in a separate reaction stage; however, it can also be employed if, for reasons of heat removal or for other reasons (cf., for example, DE-A 199 02 562), the conversion is spread over a plurality of reactors connected in series; an example of a heterogeneously catalyzed gas phase partial oxidation which is frequently carried out in two stages is the partial oxidation of propylene to acrylic acid; in the first reaction stage, the propylene is oxidized to acrolein and, in the second reaction stage, the acrolein to acrylic acid; correspondingly, the preparation of methacrylic acid is usually carried out in two stages starting from isobutene; however, when suitable catalyst charges are used, both aforementioned partial oxidations can also be carried out in one stage (both steps in one and the same fixed catalyst bed, superimposed on one another in one reactor)) heterogeneously catalyzed gas phase partial oxidation of at least one organic compound when the target product is removed more or less selectively (for example by absorption into a suitable solvent) from the product gas mixture. In general, it consists predominantly of the inert diluent gases used for the partial oxidation, and also of steam typically formed as a by-product in the partial oxidation or added as a diluent gas and carbon oxides formed by undesired full oxidation. In some cases, it also comprises small amounts of oxygen which has not been consumed in the partial oxidation (residual oxygen) and/or unconverted organic starting compounds.

The steam formed as a by-product ensures in most cases that the partial oxidation proceeds without significant changes in volume of the reaction gas mixture.

According to the above, the inert diluent gas used in most heterogeneously catalyzed gas phase partial oxidations of organic compounds consists of ≧90% by volume, frequently of ≧95% by volume, of N₂, H₂O and/or CO₂, and thus substantially of uncombustible inert diluent gases.

The inert diluent gases used are firstly helpful in taking up the heat of reaction and secondly ensure safe operation of the heterogeneously catalyzed gas phase partial oxidation of an organic compound by keeping the reaction gas mixture outside the explosion range. In heterogeneously catalyzed gas phase partial oxidations of unsaturated organic compounds, it is frequently also possible to use saturated hydrocarbons, i.e. combustible gases, as inert diluent gases. The use of inert diluent gases with elevated specific molar heat is advantageous.

Owing to a multitude of possible parallel and/or subsequent reactions, with regard to a very selective conversion of the organic starting compound to be oxidized partially to the desired target product, the measures of use of catalysts and inert diluent gas are normally insufficient for adequate thermal control of the gas phase partial oxidation. Instead, it is necessary for a very selective performance of a heterogeneously catalyzed gas phase partial oxidation in the fixed catalyst bed to additionally control the profile of the reaction temperature in the fixed catalyst bed by further measures. These additional measures generally also have an advantageous effect on the lifetime of the fixed catalyst bed. This is because it is common knowledge that a heterogeneously catalyzed gas phase partial oxidation of an organic starting compound in the fixed catalyst bed can be operated essentially continuously over prolonged periods (the lifetime) over one and the same fixed catalyst beds, and the reaction conditions can be kept essentially constant when the occurrence of excessively high temperatures in the fixed catalyst bed is largely avoided (cf., for example, DE-A 103 51 269).

As such an additional measure, an exothermic heterogeneously catalyzed gas phase partial oxidation is normally performed in a tube bundle reactor. The fixed catalyst bed is present in the tubes (reaction tubes or catalyst tubes) of the tube bundle reactor, and the reaction gas mixture is conducted through the reaction tubes thus charged. The heat of reaction is removed in a manner appropriate to the aim by conducting at least one fluid (liquid and/or gaseous) heat carrier (generally a salt melt or a liquid metal or a boiling liquid or a hot gas phase) through the space surrounding the reaction tubes in the tube bundle reactor (surrounding space). It is conducted with an entrance temperature Tⁱⁿ into the surrounding space and conducted back out of the surrounding space with an exit temperature T^{out}, where T^{out}>Tⁱⁿ (cf., for example, EP-A 700 714 and EP-A 700 893). In principle, the surrounding space can also be segmented by appropriate separating walls, and an essentially separate (the separating walls may be permeable to a small extent and enable communication of the separately supplied heat carriers) fluid heat carriers may be conducted through each of the surrounding space segments, for which T^{out}>Tⁱⁿ is fulfilled. Such a tube bundle reactor type is referred to as a multizone tube bundle reactor or, also in shortened form, simply as a multizone reactor (cf., for example, DE-A 199 27 624, DE-A 199 48 523, WO 00/53557, DE-A 199 48 248, WO 00/53558, WO 2004/085365, WO 2004/085363, WO 2004/085367, WO 2004/085369, WO 2004/085370, WO 2004/085362, EP-A 1 159 247, EP-A 1 159 246, EP-A 1 159 248, EP-A 1 106 598, WO 2005/021149, US-A 2005/0049435, WO 2004/007064, WO 05/063673, WO 05/063674, DE-A 10 2004 025 445, European application 06100535.1 and the literature cited in these documents), and an individual surrounding space segment is referred to as the temperature zone of the tube bundle reactor.

Frequently, an exothermic heterogeneously catalyzed partial gas phase oxidation of an organic starting compound to an organic target compound is operated under essentially stable operating conditions (the gas phase oxidation is in an essentially steady operating state) over a prolonged period (several days or weeks).

In other words, in an operating state I, a starting reaction gas mixture comprising the organic target compound, molecular oxygen and at least one inert diluent gas is conducted, essentially while retaining its composition over time and with essentially constant loading of the fixed catalyst bed disposed in the reaction tubes over time (the reaction tubes are normally all filled substantially uniformly), with a load L^{I} of the organic starting compound through a fixed catalyst bed disposed in the tubes of a tube bundle reactor, in whose surrounding space at least one fluid heat carrier is conducted in with an essentially stable entrance temperature T_{I}ⁱⁿ and conducted out of it again with an essentially stable exit temperature T_{I}^{out}. The selected operating conditions are adjusted primarily to the space-time yield of organic target compound required from the production plant in the production period in question, and to the operational age of the fixed catalyst bed (a deactivation of the fixed catalyst bed which accompanies prolonged operating time can be counteracted, for example, by measures such as increasing the working pressure (cf., for example, DE-A 10 2004 025 445) and/or T_{I}ⁱⁿ (cf. DE-A 103 51 269).

In many cases, the market demand for an organic target compound is, however, not a stable parameter but rather fluctuates over prolonged periods.

For example, it can rise sharply. Instead of reacting to such a rise in market demand with an additional production plant, it is also possible to react to it with an increase in the space-time yield of target product in already existing production plants. This is possible, for example, by leaving the operating state I and changing to an operating state II which is characterized both by a higher value L^{II} (>L^{I}) for the load of the fixed catalyst bed with the organic starting compound and by a higher entrance temperature T_{II}ⁱⁿ (>T_{I}ⁱⁿ) of the at least one fluid heat carrier into the surrounding space. The increase in the entrance temperature Tⁱⁿ of the fluid heat carrier is for the purpose of converting the same molar fraction of organic starting compound with shortened reactant residence time in the fixed catalyst bed as a result of the increase in load, and hence of increasing the space-time yield of target product.

EP-A 1 695 954 teaches undertaking the change from operating state I to operating state II in such a way that, while retaining the value T_{I}ⁱⁿ for the entrance temperature of the fluid heat carrier, first increasing the load L^{I} to the value L^{II} and only thereafter increasing the temperature T_{I}ⁱⁿ in small steps to the temperature T_{II}ⁱⁿ. As an advantage of this procedure, EP-A 1 695 954 asserts that the increase in the so-called hotspot temperature of the fixed catalyst bed which is associated with the change in operating state passes through a very low maximum when the change is made.

The hotspot temperature T^{max} refers to the highest temperature which occurs within a temperature zone in flow direction of the reaction gas mixture in the fixed catalyst bed. This highest temperature of the fixed catalyst bed in the particular temperature zone is above the accompanying value for Tⁱⁿ for the particular temperature zone. T^{max} is essentially identical to the highest reaction temperature value which occurs in the corresponding temperature zone. The difference between T^{max} and Tⁱⁿ is referred to as the hotspot expansion of the particular temperature zone.

However, a disadvantage of the procedure proposed in EP-A 1 695 954 is that, after the increase from L^{I} to L^{II} up to the time at which Tⁱⁿ has attained the value required for T_{II}ⁱⁿ when changing from T_{I}ⁱⁿ to T_{II}ⁱⁿ, the value for Tⁱⁿ lags behind the increased value L^{II} for the loading of the fixed catalyst bed of the organic starting compound. In other words, as the reaction gas input mixture passes through the fixed catalyst bed, an increased proportion of the organic starting compound is not converted to the desired target product. After the removal of the target product from the product gas mixture, a residual gas with increased proportion of the organic starting compound thus remains. Since the residual gas can be recycled at best partially as cycle gas into the partial oxidation (otherwise, the inert gas content of the reaction gas input mixture accumulates constantly) and is otherwise normally disposed of (for example incinerated), a significant loss of product of value results along the procedure recommended in EP-A 1695954. Furthermore, when the organic starting compound, as in the case of acrolein (for example in the case of its partial oxidation to acrylic acid), is a compound which has a marked tendency to undesired spontaneous free-radical polymerization, acrolein unconverted in the, for example, acrolein partial oxidation can lead to undesired polymer formation to a considerable degree in the course of the target product (for example acrylic acid) removal and necessitate an interruption in the target product removal, in order to free the separating apparatus (for example column) used for this purpose of undesired polymer.

It is therefore an object of the present invention to provide a process for changing the operating state of an exothermic heterogeneously catalyzed partial gas phase oxidation of an organic starting compound which has the indicated disadvantages of the procedure recommended in the prior art at worst to a reduced extent.

Accordingly, a process has been found for operating an exothermic heterogeneously catalyzed partial gas phase oxidation of an organic starting compound to an organic target compound in different operating states I and II, in which a reaction gas input mixture comprising the organic starting compound, molecular oxygen and at least one inert diluent gas is passed through a fixed catalyst bed disposed in the tubes of a tube bundle reactor to obtain a product gas mixture comprising the organic target compound, and the reaction temperature in the fixed catalyst bed disposed in the tubes is adjusted by conducting at least one fluid heat carrier into the space surrounding the tubes of the tube bundle reactor which comprise the fixed catalyst bed with an entrance temperature Tⁱⁿ and out of it again with an exit temperature T^{out}>Tⁱⁿ, and the fixed catalyst bed is loaded in the operating state I at an entrance temperature T_{I}ⁱⁿ with a load L^{I} of the organic starting compound and, in the operating state II, at an entrance temperature T_{II}ⁱⁿ with a load L^{II} of the organic starting compound, with the proviso that L^{II}>L^{I} and T_{II}ⁱⁿ>T_{I}ⁱⁿ, which comprises changing from operating state I to operating state II by first increasing the entrance temperature T_{I}ⁱⁿ to the value T_{II}ⁱⁿ and then increasing the load of the fixed catalyst bed with the organic starting compound from the value L^{I} to the value L^{II}.

Advantageously in accordance with the invention, the change from T_{I}ⁱⁿ to T_{II}ⁱⁿ will be performed as a succession of small temperature increase steps. After the performance of one temperature increase step, the new operating conditions will first be retained in each case for a certain time (typically over an operating period of at least 5 minutes, or at least 10 minutes, i.e., for example, from 5 minutes to 360 minutes, frequently over an operating period of from 10 minutes to 240 minutes and in many cases over an operating period of from 15 minutes to 120 minutes, or from 20 minutes to 60 minutes), before the next temperature increase step regarding Tⁱⁿ is implemented. The lower the individual temperature increase step is selected in each case, the more moderate the associated change in the hotspot temperature which occurs. Appropriately from an application point of view, one temperature increase step for Tⁱⁿ is not less than 0.1° C. Normally, one temperature increase step for Tⁱⁿ will, though, not be more than 10° C., generally not more than 5° C. and, in a manner particularly appropriate from an application point of view, not more than 2° C. Typical temperature increase steps for Tⁱⁿ are therefore 0.2° C., or 0.3° C., or 0.4° C., or 0.5° C., or 0.6° C., or 0.7° C., or 0.8° C., or 0.9° C., or 1° C. It will be appreciated that one temperature increase step for Tⁱⁿ may also be 1.5° C. In a manner favorable from an application point of view, one temperature increase step for Tⁱⁿ will be from 0.1 to 1° C., preferably from 0.2 to 0.8° C., more preferably from 0.3 to 0.7° C. and most preferably from 0.4 to 0.6° C.

In an entirely corresponding manner, the increase in the load of the fixed catalyst bed with the organic starting compound from the value L^{I} to the value L^{II} will, advantageously in accordance with the invention, be performed as a succession of small load increase steps. After the performance of one load increase step, the new operating conditions will first be retained in each case for certain time (typically over an operating period of at least 5 minutes, or at least 10 minutes, i.e., for example, from 5 minutes to 360 minutes, frequently over an operating period of from 10 minutes to 240 minutes and in many cases over an operating period of from 15 minutes to 120 minutes, or from 20 minutes to 60 minutes), before the next load increase step regarding the load of the fixed catalyst bed with the organic starting compound is implemented. The lower the individual load temperature increase step is selected in each case, the more moderate the associated change in the hotspot load which occurs. Based on the value of L^{I}, a load increase step, appropriately from an application point of view is not less than 1%. Normally, a load increase step on a corresponding basis will, though, not be more than 50%, generally not more than 40%, appropriately from an application point of view not more than 30% and particularly appropriately not more than 20%. Typical load increase steps on the same basis are therefore 5%, or 10%, or 15%, or 20%, or 25%, or 30%, or 35%, or 40%, or 45%, or 50%. In a manner favorable from an application point of view, a load increase step on the same basis regarding the load of the fixed catalyst bed with the organic starting compound will be from 1% to 50%, or from 2% to 45%, or from 5% to 40%, or from 10% to 35%, or from 15% to 30%, or from 20% to 30%, or 25% to 30%.

Very particularly advantageously in accordance with the invention, the procedure for increasing the space-time yield of the desired target compound to satisfy an increased market demand will be to perform the inventive procedure several times in succession. In other words, starting from the operating state I (referred to hereinafter as the original operating state I), T_{I}ⁱⁿ is first increased by only one temperature increase step (for example from 0.1 to 10° C., preferably from 0.1 to 2° C. or to 1° C., frequently by 0.5° C.). The result is a new entrance temperature T_{II}ⁱⁿ. The process is then operated for a certain time under the new operating conditions (for example from 5 minutes to 360 minutes, or from 10 minutes to 240 minutes, or from 15 minutes to 120 minutes, preferably from 10 minutes to 60 minutes). Subsequently, the load of the fixed catalyst bed with the organic starting compound is increased from the value L^{I} to the value L^{II} which essentially corresponds to the new entrance temperature T_{II}ⁱⁿ (normally in such a way that the conversion of the organic starting compound in the operating state II which results in this way is essentially the same as that in the previous operating state I).

The process is then operated under the new operating conditions for a certain time (for example from 5 minutes to 360 minutes, or from 10 minutes to 240 minutes, or from 15 minutes to 120 minutes, preferably from 10 minutes to 60 minutes).

The operating state II which has then been assumed forms the new starting operating state I from which the procedure is the same as from the original operating state I, etc. After repeated successive use of the inventive procedure in such a way, a final operating state II is finally achieved, which is capable of satisfying the increased market demand for the desired target product. Starting from the original operating state I, a route is taken to the final operating state II in which the associated change in the hotspot temperature is moderated to a very particularly marked degree. The smaller the individual temperature increase step selected in each case in this procedure too, the more true this is.

The difference between L^{I} and L^{II} in the process according to the invention can in principle vary widely. In many cases, the ratio V of L^{I} to L^{II} will, however, be ≦2.

In other words, V may be, for example, >1 and ≦2, or ≧1.05 and ≦1.90, or ≧1.10 and ≦1.80, or ≧1.20 and ≦1.60, or ≧1.25 and ≦1.50, or ≧1.30 and ≦1.40.

In a simple manner, the increase required in accordance with the invention of the load L^{I} to the value L^{II} can be effected with essentially uniform composition of the reaction gas input mixture by increasing the load of the fixed catalyst bed with reaction gas input mixture in a corresponding manner (i.e. in the same ratio as L^{II}/L^{I}). Alternatively, the increase required in accordance with the invention of the load L^{I} to the value L^{II} can also be effected by, with essentially constant load of the fixed catalyst bed with reaction gas input mixture, increasing the proportion of the organic starting compound in the reaction gas input mixture in a corresponding manner. In such a procedure, the proportion of the molecular oxygen in the reaction gas input mixture will normally also be increased in a corresponding manner, so that the molar ratio R of organic starting compounds to molecular oxygen in the reaction gas input mixture of the operating state II is essentially equal to that in the reaction gas input mixture of the operating state I (in this case, the inert gas proportion in the reaction gas input mixture decreases when changing from operating state I to operating state II). Appropriately in accordance with the invention, the increase in the proportion of the molecular oxygen and the increase in the proportion of the organic starting compound in the reaction gas input mixture will be undertaken synchronously (i.e. essentially at the same time)(advantageously from an application and safety point of view, the increase in the stream of the source of the organic starting compound is conducted slightly earlier and acts as a control variable for the subsequent increase in the stream of the oxygen source). It will be appreciated that the two aforementioned methods for increasing L^{I} may also be employed in combination. Normally, in that case too, the value for R in the reaction gas input mixture of the operating state I will essentially be retained when changing to the operating state II.

In other words, the procedure will frequently be to keep the cycle gas stream (the cycle gas amount/time) for the reaction gas mixture input stream essentially constant (or adapted beforehand or afterward to the increase in the reactant feed streams) and to increase the feed stream of the organic starting compound and the oxygen source (generally air) for the reaction gas mixture input stream.

When the process according to the invention is the second reaction stage of a two-stage heterogeneously catalyzed partial oxidation, the target product of the first reaction stage is normally the organic starting compound for the second reaction stage (cf., for example, the two-stage heterogeneously catalyzed partial gas phase oxidation of propylene to acrylic acid; the acrolein formed in the first reaction stage from the organic starting compound propylene forms the organic starting compound for the acrylic acid formed in the second reaction stage).

The reaction gas input mixture fed to the second reaction stage is regularly the product gas mixture of the first reaction stage which has been cooled beforehand if appropriate and has been supplemented if appropriate with molecular oxygen and/or inert gas.

When each of the two reaction stages is performed in a tube bundle reactor (for example each in one (for example one as shown in FIG. 1 of EP-A 1 695 954) of two successive tube bundle reactors in each case, or each in at least one temperature zone in each case or a multizone tube bundle reactor (of a so-called single reactor)), the process according to the invention will be employed synchronously both on the first and on the second reaction stage.

In other words, the process according to the invention also comprises a process for operating a two-stage exothermic heterogenously catalyzed partial gas phase oxidation of a first organic starting compound to an organic final target compound in different operating states I and II, in which a first reaction gas input mixture comprising the first organic starting compound, molecular oxygen and at least one inert diluent gas is passed through a first fixed catalyst bed disposed in the tubes of a tube bundle reactor to obtain a first product gas mixture comprising an organic intermediate target compound, and the reaction temperature in the first fixed catalyst bed is adjusted by conducting at least one fluid heat carrier into the space surrounding the tubes of this tube bundle reactor which comprise the first fixed catalyst bed with an entrance temperature T^{1,in} and out of it again with an exit temperature T^{1,out}>T^{1,in}, and the first product gas mixture which has been cooled beforehand if appropriate and supplemented with molecular oxygen and inert gas as a secondary gas additive (secondary gas addition) if appropriate is conducted as a second reaction gas input mixture comprising molecular oxygen, at least one inert diluent gas and the organic intermediate target compound as a second organic starting compound to obtain a second product gas mixture comprising the organic final target compound through a second fixed catalyst bed which is different from the first fixed catalyst bed and is disposed in the tubes of a tube bundle reactor, and the reaction temperature in the second fixed catalyst bed is adjusted by conducting at least one second fluid heat carrier with an entrance temperature T^{2,in} into the space surrounding the tubes of this tube bundle reactor which comprise the second fixed catalyst bed and out of it again with an exit temperature T^{2,out}>T^{2,in}and, in the operating state I, the first fixed catalyst bed is loaded at an entrance temperature T_{I}^{1,in} in with a load L^{1,I} of the first organic starting compound and the second fixed catalyst bed at an entrance temperature T_{I}^{2,in} with a load L^{2,I} of the second organic starting compound, and, in the operating state II, the first fixed catalyst bed is loaded at an entrance temperature T_{II}^{1,in} with a load L^{1,II} of the first organic starting compound and the second fixed catalyst bed at an entrance temperature T_{II}^{2,in} with a load L^{2,II} of the second organic starting compound, with the proviso that L^{1,II}>L^{1,I}, T_{II}^{1,in}>T_{I}^{1,in}, L^{2,II}>L^{2,I} and T_{II}^{2,in}>T_{I}^{2,in}, which comprises changing from operating state I to operating state II by first increasing the entrance temperature T_{I}^{1,in} to the value T_{II}^{1,in} and the entrance temperature T_{I}^{2,in} to the value T_{II}^{2,in}, and then increasing the loading of the first fixed catalyst bed with the first organic starting compound from the value L^{1,I} to the value L^{1,II}, and, as a consequent effect of the latter measure above and of any additionally changed secondary gas addition, increasing the loading of the second fixed catalyst bed from the value L^{2,I} to the value L^{2,II}.

The aforementioned is especially true when the two-stage exothermic heterogeneously catalyzed partial gas phase oxidation is the two-stage exothermic heterogeneously catalyzed partial gas phase oxidation of propylene to acrylic acid, or an exothermic two-stage heterogeneously catalyzed partial oxidation of isobutene to methacrylic acid.

In the former case, the first organic starting compound is propylene, the intermediate target compound (the second organic starting compound) is acrolein and the final target compound is acrylic acid.

In principle, in the above-described two-stage partial oxidation, the increase in the entrance temperature T_{I}^{2,in} to the value T_{II}^{2,in} and the increase in the entrance temperature T_{I}^{2,in} to the value T_{II}^{2,in} will be performed essentially synchronously (simultaneously).

To avoid increased proportions of unconverted intermediate target product (of unconverted intermediate target compound) in the second product gas mixture of the two-stage heterogeneously catalyzed exothermic gas phase partial oxidation, it is, however, advantageous to allow the increase of T_{I}^{1,in} to always lag somewhat behind the increase of T_{I}^{2,in} in time (the second reaction stage is thus actually already prepared for the slightly increased formation rate of intermediate target compound which is established as a result of the increase of T_{I}^{1,in} the first reaction stage even with initially constant load of the first fixed catalyst bed with the first organic starting compound). In principle, the present invention also encompasses those procedures in two-stage heterogeneously catalyzed exothermic gas phase partial oxidations in which, in a change from an operating state I to an operating state II, the increase in the inlet temperatures T_{I}^{1,n} and T_{II}^{2,in} is always undertaken in such a way that the increase of T_{I}^{2,in} always lags somewhat behind the increase of T_{I}^{1,in} in time.

Advantageously in accordance with the invention, in a two-stage procedure, the change from T_{I}^{1,in} to T_{II}^{1,in} and the change of T_{I}^{2,in} to T_{II}^{2,in} will be performed in the same manner as in the one-stage procedure, preferably as a succession of small temperature increase steps.

In other words, after the performance of a temperature increase step essentially synchronously in both reaction stages, the new operating conditions will first be retained for a certain time in each case (typically over an operating period of at least 5 minutes, or at least 10 minutes, i.e., for example, from 5 minutes to 360 minutes, frequently over an operating period of from 15 minutes to 120 minutes, or from 20 minutes to 60 minutes), before the next temperature increase step, which is to be performed essentially synchronously for both reaction stages, regarding the particular entrance temperature of the particular fluid heat carrier is implemented. The lower the individual temperature increase step is selected in each case, the more moderate the associated changes in the particular hotspot temperature in both temperature zones which occur.

Appropriately from an application point of view, one temperature increase step for the particular Tⁱⁿ, in a two-stage procedure too, is not less than 0.1° C. Normally, such a temperature increase step will, though, not be more than 10° C., generally not more than 5° C. and, in a manner particularly appropriate from an application point of view, not more than 2° C. Typical temperature increase steps for the particular Tⁱⁿ are therefore 0.2° C., or 0.3° C., or 0.4° C., or 0.5° C., or 0.6° C., or 0.7° C., or 0.8° C., or 0.9° C., or 1° C. Of course, a temperature increase step for the particular Tⁱⁿ may also be 1.5° C. In a manner favorable from an application point of view, one temperature increase step for T_{I}^{1,in} or T_{I}^{2,in} will be from 0.1 to 1° C., preferably from 0.2 to 0.8° C., more preferably from 0.3 to 0.7° C. and most preferably from 0.4 to 0.6° C.

In a completely corresponding manner, in a two-stage procedure, the load of the first fixed catalyst bed with the first organic starting compound from the value L^{1,I} to the value L^{1,II} will, advantageously in accordance with the invention, be performed as a succession of small load increase steps. After the performance of one load increase step, the new operating conditions will first be retained for a certain time in each case (typically over an operating period of at least 5 minutes, or at least 10 minutes, i.e., for example, from 5 minutes to 360 minutes, frequently over an operating period of from 10 minutes to 240 minutes and in many cases over an operating period of from 15 minutes to 120 minutes, or from 20 minutes to 60 minutes) before the next load increase step regarding the load of the first fixed catalyst bed with the first organic starting compound is implemented. The lower the individual load increase step is selected in each case, the more moderate the associated change in the accompanying hotspot temperature which occurs. Based on the value of L^{1,I} a load increase step, appropriately from an application point of view, is not less than 1%. Normally, a load increase step, on a corresponding basis, will, though, not be more than 50%, generally not more than 40%, appropriately from an application point of view not more than 30% and particularly appropriately not more than 20%.

Typical load increase steps on the same basis are therefore 5%, or 10%, or 15%, or 20%, or 25%, or 30%, or 35%, or 40%, or 45%, or 50%. In a manner favorable from an application point of view, a load increase step on the same basis, regarding the load of the first fixed catalyst bed with the first organic starting compound, will be from 1% to 50%, or from 2% to 45%, or from 5% to 40%, or from 10% to 35%, or from 15% to 30%, or from 20% to 30%, or from 25% to 30%.

The increase in the load L^{1,II} to the value L^{1,II} which is to be performed in accordance with the invention in a two-stage heterogeneously catalyzed exothermic partial oxidation of a first organic starting compound which is to be operated as described above can be effected in a simple manner by, with essentially constant composition of the reaction gas input mixture comprising the first organic starting compound, increasing the load of the first fixed catalyst bed with reaction gas input mixture in a corresponding manner (i.e. in the same ratio as L^{1,II}/L^{1,I}).

Alternatively, the load L^{1,I} can also be increased here to the value L^{1,II} by, with essentially constant load of the first fixed catalyst bed with the reaction gas input mixture comprising the first organic starting compound, increasing the proportion of the first organic starting compound in the reaction gas input mixture in a corresponding manner. In such a procedure, the proportion of the molecular oxygen in the first reaction gas input mixture comprising the first organic compound will normally also be increased in a corresponding manner, so that the molar ratio R of the first organic starting compound to molecular oxygen in the first reaction gas input mixture of operating state II corresponds essentially to that in the first reaction gas input mixture of operating state I (in this case, the inert gas content in the first reaction gas input mixture decreases at the transition from operating state I to operating state II). Appropriately in accordance with the invention, the increase in the content of molecular oxygen and the increase in the content of the first organic starting compound in the first reaction gas input mixture will be undertaken synchronously (i.e. essentially simultaneously); here too, the increase in the content of the first organic starting compound, appropriately from an application point of view, again conducts the increase in the content of molecular oxygen.

It will be appreciated that the two aforementioned methods for increasing L^{1,I} may also be employed in combination. Of course, in that case too, the value for R in the first reaction gas input mixture of operating state I will normally essentially be retained at the transition to operating state II.

Alternatively, in the case of an increase of L^{1,I}, the procedure may also be to keep the residual oxygen content in the first product gas mixture (in the product gas mixture of the first reaction stage) stable in operating states I, II (for example at a value in the range of from 1 to 9% by volume). Any reductions in conversion which arise therefrom (owing to an increasing R) can be captured by means of a corresponding increase in T_{II}^{1,in}.

In other words, the procedure will frequently be to keep the cycle gas stream (the amount of cycle gas/time) for the reaction gas mixture input stream essentially constant (or adapted beforehand or afterward to the increase in the reactant feed streams), and to increase the feed stream of the first organic starting compound and of the oxygen source (generally air) for the first reaction gas mixture input stream.

When no supplementary molecular oxygen and/or inert gas is added (no secondary gas is added) to the first product gas mixture of a two-stage heterogeneously catalyzed exothermic partial oxidation to be performed as described before its further use as the second reaction gas input mixture, as is necessarily the case, for example, in the case of performance of the two-stage partial oxidation in a single reactor (cf., for example, FIG. 4 of EP-A 1 695 954), the corresponding increase of L^{2,I} to L^{2,II} normally automatically also accompanies an increase of L^{1,I} L^{1,II}. The oxygen demand in the second reaction stage is already taken account of by a corresponding oxygen content in the first reaction gas input mixture. When secondary gas is supplemented into the first product gas mixture between the two reaction stages, this is normally done in such a way that the increase of L^{1,I} to L^{1,II} is accompanied by the desired increase (the corresponding increase step) of L^{2,I} to L^{2,II}, and, at the same time, the molar ratio R of second organic starting compound to molecular oxygen in the second reaction gas input mixture of operating state II is essentially the same as that in the second reaction gas input mixture of operating state I.

Very particularly advantageously in accordance with the invention, the space-time yield of the desired final target compound will be increased to satisfy an increased market demand in a two-stage exothermic heterogeneously catalyzed partial oxidation to be performed as described in such a way that the inventive procedure is performed several times in succession.

Alternatively, a secondary oxygen feeding (for example as secondary air) can also be undertaken in such a way that the residual oxygen content in the product gas mixture of the second reaction stage is kept stable in operating states I, II (for example at a value in the range of from 1 to 4% by volume). Any reductions in conversion which arise therefrom (owing to an increasing R) can be captured by means of a corresponding increase of T_{II}^{2,in}.

In other words, starting from operating state I (referred to hereinafter as the original operating state I), T_{I}^{1,in} and T_{I}^{2,in} are first increased as described by only one temperature increase step (for example from 0.1 to 10° C., preferably from 0.2 to 5° C., more preferably from 0.3 to 2° C., most preferably from 0.4 to 1.5° C. and in many cases from 0.5 to 1° C.). The result is the new entrance temperatures T_{II}^{1,in} and T_{II}^{2,in}. The process is then operated under the new operating conditions for a certain time (for example over a period of at least 5 minutes, or at least 10 minutes, i.e., for example, from 5 minutes to 360 minutes, frequently over an operating period of from 10 minutes to 240 minutes and in many cases over an operating period of from 15 minutes to 120 minutes, or from 20 minutes to 60 minutes. Subsequently, the load of the first fixed catalyst bed with the first organic starting compound is increased from the value L^{1,I} to the value L^{1,II} which essentially corresponds to the new entrance temperature T_{II}^{1,in} (normally in such a way that the conversion of the first organic starting compound in the operating state II which results in this way is largely the same as that in the preceding operating state I). As a consequence of this measure, and also of any additionally changed secondary gas addition, there is normally an accompanying increase (an increase step) in the load of the second fixed catalyst bed with the second organic target compound from the value L^{2,I} to the value L^{2,II}, which corresponds essentially to the new entrance temperature T_{II}^{2,in} (normally in such a way that the conversion of the second organic starting compound in the operating state II which results in this way corresponds largely to that in the preceding operating state I). The process is then operated under the new operating conditions for a certain time (for example over a period of at least 5 minutes, or at least 10 minutes, i.e., for example, from 5 minutes to 360 minutes, frequently over an operating period of from 10 minutes to 240 minutes and in many cases over an operating period of from 15 minutes to 120 minutes, or from 20 minutes to 60 minutes). The operating state II which is then assumed forms the new starting operating state I, from which the same procedure is performed as from the original operating state I, etc. After such repeated successive use of the inventive procedure for two-stage heterogeneously catalyzed exothermic gas phase partial oxidations, a final operating state II which is capable of satisfying the increased market demand for the desired target product is finally attained.

Starting from the original operating state I, a path to the final operating state II is taken in which the accompanying change in the particular hotspot temperature is moderated to a very particularly marked degree. In this procedure too, the smaller the individual temperature increase step selected in each case, the more true this is.

The difference between L^{1,I} and L^{1,II} may in principle vary widely in the two-stage procedure described. In many cases, the ratio V1 of L^{1,II} to L^{1,I} will, however, be ≦2.

In other words, V1 may, for example, be >1 and ≦2, or ≧1.05 and ≦1.90, or ≧1.10 and ≦1.80, or ≧1.20 and ≦1.60, or ≧1.25 and ≦1.50, or ≧1.30 and ≦1.40. The same applies for the ratio V2 of L^{2,II} to L^{2,I}.

However, it will be appreciated that the inventive procedure may be employed not only for the purpose of adjustment to changed market situations. Instead, the inventive procedure may also be employed advantageously in the startup of a production plant. In that case, an entrance temperature T_{I}ⁱⁿ* roughly suitable for the envisaged space-time yield of target product, is generally predefined, whose magnitude is generally, however, a few ° C. (generally not more than 15° C. and usually not more than 10° C.) below the entrance temperature T_{I}ⁱⁿ which corresponds to the load L^{I}, corresponding to the planned space-time yield of target product, of the fixed catalyst bed of predetermined activity with the organic starting compound (when the fixed catalyst bed is being restarted on completion of regeneration, T_{I}ⁱⁿ* will in every case be below the last value of Tin before the performance of the regeneration (for example according to DE-A 103 51 269)).

The fixed catalyst bed is then loaded with reaction gas input mixture. The selected load L of the fixed catalyst bed with the organic starting compound is about 60% of that value which, as the specified load, is envisaged as corresponding to T_{I}ⁱⁿ*. With essentially constant composition of the reaction gas input mixture (preferably as described in DE-A 103 37 788), the load of the fixed catalyst bed with the organic starting compound is increased stepwise as described until, based on single pass of the reaction gas input mixture through the fixed catalyst bed, the conversion of the organic starting compound attains its specified conversion corresponding to the envisaged space-time yield.

Starting from this operating state I, T_{I}ⁱⁿ and L^{I} are then increased in the inventive manner to such an extent that the space-time yield attains its planned specified value. In the case of a two-stage exothermic heterogeneously catalyzed gas phase partial oxidation, the procedure is entirely analogous.

The values for L^{I} and L^{II} may, in the process according to the invention (depending on the specific heterogeneously catalyzed exothermic gas phase partial oxidation performed), extend over a wide range. In other words, L^{I}, L^{II} may be from 5 or 100 to 5000 l (STP)/I·h, or from 20 or 150 to 4000 l (STP)/I·h, or from 50 or 200 to 2000 I (STP)/I·h, or from 250 to 1000 l (STP)/I·h.

For successful use of the inventive procedure, it is not indispensable, but it is advantageous, when the conversion C^{A} based on single pass of the particular reaction gas input mixture through the fixed catalyst bed (in mol % based on the molar amount of the organic starting compound present in the reaction gas input mixture) of the organic starting compound present in the reaction gas input mixture in operating states I (C^{A,I}) and II (C^{A,II}) is essentially the same. Favorably in accordance with the invention, the magnitude of the difference between C^{A,II} and C^{A,I} is less than 5 mol %, better less than 3 mol %. even better less than 1 mol %, particularly favorably less than 0.5 mol % and at best less than 0.1 mol %.

The process according to the invention is advantageous in accordance with the invention when both C^{A,I} and C^{A,II} are ≧50 mol %, or ≧75 mol %, or ≧90 mol %, or ≧95 mol % or ≧98 mol %, or ≧99 mol %, or ≧99.5 mol %, or ≧99.9 mol %.

When the heterogeneously catalyzed partial exothermic gas phase oxidation to be performed in accordance with the invention is the heterogeneously catalyzed partial gas phase oxidation of propylene to acrolein and/or acrylic acid (as an independent process or as the first reaction stage in a two-stage partial oxidation of propylene to acrylic acid (first reaction stage: propylene→acrolein)), it is advantageous in accordance with the invention when the propylene content in the product gas mixture, both in operating state I and in operating state II, does not exceed the value of 10 000 ppm by weight, preferably 6000 ppm by weight and more preferably 4000 or 2000 ppm by weight. It is even better when, in the process according to the invention, the particular aforementioned propylene content in the product gas mixture is not exceeded not only in operating states I and II but also in all transitional states in the transition from operating state I to operating state II.

When the heterogeneously catalyzed partial exothermic gas phase oxidation to be performed in accordance with the invention is the heterogeneously catalyzed partial gas phase oxidation of acrolein to acrylic acid (as an independent process or as the second reaction stage in a two-stage partial oxidation of propylene to acrylic acid (second reaction stage: acrolein→acrylic acid)), it is advantageous in accordance with the invention when the acrolein content in the product gas mixture, both in operating state I and in operating state II, does not exceed the value of 1500 ppm by weight, preferably 600 ppm by weight and more preferably 350 ppm by weight. It is even better when, in the process according to the invention, the particular aforementioned acrolein content in the product gas mixture is not exceeded not only in operating states I and II but also in all transitional states in the transition from operating state I to operating state II.

The process according to the invention is suitable for a heterogeneously catalyzed exothermic gas phase fixed bed partial oxidation of propene to acrolein especially when the catalysts used are those whose active composition is a multielement oxide which comprises the elements molybdenum and/or tungsten, and also at least one of the elements bismuth, tellurium, antimony, tin and copper, or is a multimetal oxide comprising the elements Mo, Bi and Fe. Multimetal oxide compositions of the aforementioned type which comprise Mo, Bi and Fe and are particularly suitable in accordance with the invention are in particular the multimetal oxide compositions comprising Mo, Bi and Fe which are disclosed in DE-A 103 44 149 and in DE-A 103 44 264. These are in particular also the multimetal oxide active compositions of the general formula I of DE-A 199 55 176, the multimetal oxide active compositions of the general formula I of DE-A 199 48 523, the multimetal oxide active compositions of the general formulae I, II and III of DE-A 101 01 695, the multimetal oxide active compositions of the general formulae I, II and III of DE-A 199 48 248 and the multimetal oxide active compositions of the general formulae I, II and III of DE-A 199 55 168 and also the multimetal oxide active compositions specified in EP-A 700 714.

An application of the process according to the invention is also suitable when the catalysts used for the fixed catalyst bed to be used in accordance with the invention, in the case of the partial oxidation of propylene to acrolein, are the multimetal oxide catalysts comprising Mo, Bi and Fe which are disclosed in the documents DE-A 100 46 957, DE-A 100 63 162, DE-C 33 38 380, DE-A 199 02 562, EP-A 015 565, DE-C 23 80 765, EP-A 807 465, EP-A 279 374, DE-A 33 00 044, EP-A 575 897, U.S. Pat. No. 4,438,217, DE-A 198 55 913, WO 98/24746, DE-A 197 46 210 (those of the general formula II), JP-A 91/294 239, EP-A 293 224 and EP-A 700 714. This applies in particular to the exemplary embodiments in these documents, and among these particular preference is given to those of EP-A 015 565, EP-A 575 897, DE-A 197 46 210 and DE-A 198 55 913. Particular emphasis is given in this context to a catalyst according to example 1c from EP-A 015 565 and also to a catalyst to be prepared in a corresponding manner but whose active composition has the composition Mo₁₂Ni_{6.5}Zn₂Fe₂Bi₁P_{0.0065}K_{0.06}O_{X}·10 SiO₂. Emphasis is also given to the example having the serial number 3 from DE-A 198 55 913 (stoichiometry: Mo₁₂Co₇Fe₃Bi_{0.6}K_{0.08}Si_{1.6}Ox) as an unsupported hollow cylinder catalyst of geometry 5 mm×3 mm×2 mm or 5 mm×2 mm×2 mm (each external diameter×height×internal diameter) and also to the unsupported multimetal oxide II catalyst according to example 1 of DE-A 197 46 210. Mention should also be made of the multimetal oxide catalysts of U.S. Pat. No. 4,438,217. The latter is true in particular when these have a hollow cylinder geometry of the dimensions 5.5 mm×3 mm×3.5 mm, or 5 mm×2 mm×2 mm, or 5 mm×3 mm×2 mm, or 6 mm×3 mm×3 mm, or 7 mm×3 mm×4 mm (each external diameter x height x internal diameter). Likewise suitable in the context of the present invention are the multimetal oxide catalysts and geometries of DE-A 101 01 695 or WO 02/062737. Also particularly suitable for the propylene to acrolein partial oxidation are the multimetal oxide compositions recommended in DE-A 10 2005 037 678 for this partial oxidation (reaction stage), especially those of the comparative examples and of the working examples of this document (the specific surface areas of the active compositions reported there are numerically correct, but the dimension should correctly be m²/g instead of cm²/g).

Also very suitable in the context of the present invention are example 1 of DE-A 100 46 957 (stoichiometry: [Bi₂W₂O₉×2WO₃]_{0.5}·[Mo₁₂Co_{5.6}Fe_{2.94}Si_{1.59}K_{0.08}Oₓ]₁) as an unsupported hollow cylinder (ring) catalyst of geometry 5 mm×3 mm×2 mm or 5 mm×2 mm×2 mm (each external diameter×length×internal diameter), and also the coated catalysts 1, 2 and 3 of DE-A 100 63 162 (stoichiometry: Mo₁₂Bi_{1.0}Fe₃Co₇Si_{1.6}K_{0.08}), except as annular coated catalysts of appropriate coating thickness and applied to support rings of geometry 5 mm×3 mm×1.5 mm or 7 mm×3 mm×1.5 mm (each external diameter x length x internal diameter).

A multitude of multimetal oxide active compositions particularly suitable for the catalysts of a propylene partial oxidation to acrolein in the context of the present invention can be encompassed by the general formula I

Mo₁₂BiₐFe_{b}X¹_{c}X²_{d}X³ₑX⁴_{f}Oₙ (I)

in which the variables are each defined as follows:
X¹=nickel and/or cobalt,
X²=thallium, an alkali metal and/or an alkaline earth metal,
X³=zinc, phosphorus, arsenic, boron, antimony, tin, cerium, lead and/or tungsten,
X⁴=silicon, aluminum, titanium and/or zirconium,
a=from 0.5 to 5,
b=from 0.01 to 5, preferably from 2 to 4,
c=from 0 to 10, preferably from 3 to 10,
d=from 0 to 2, preferably from 0.02 to 2,
e=from 0 to 8, preferably from 0 to 5,
f=from 0 to 10 and
n=a number which is determined by the valency and frequency of the elements in I other than oxygen.

They are obtainable in a manner known per se (see, for example, DE-A 40 23 239) and are customarily shaped undiluted to give spheres, rings or cylinders or else used in the form of coated catalysts, i.e. preshaped inert support bodies coated with the active composition. It will be appreciated that they may also be used as catalysts in powder form.

In principle, active compositions of the general formula I can be prepared in a simple manner by obtaining a very intimate, preferably finely divided dry mixture having a composition corresponding to their stoichiometry from suitable sources of their elemental constituents and calcining it at temperatures of from 350 to 650° C. The calcination may be effected either under inert gas or under an oxidative atmosphere, for example air (mixture of inert gas and oxygen) and also under a reducing atmosphere (for example mixture of inert gas, NH₃, CO and/or H₂). The calcination time can be from a few minutes to a few hours and typically decreases with temperature. Useful sources for the elemental constituents of the multimetal oxide active compositions I are those compounds which are already oxides and/or those compounds which can be converted to oxides by heating, at least in the presence of oxygen.

In addition to the oxides, such useful starting compounds include in particular halides, nitrates, formates, oxalates, citrates, acetates, carbonates, amine complexes, ammonium salts and/or hydroxides (compounds such as NH₄OH, (NH₄)₂CO₃, NH₄NO₃, NH₄CHO₂, CH₃COOH, NH₄CH₃CO₂ and/or ammonium oxalate which decompose and/or can be decomposed on later calcining at the latest to give compounds which are released in gaseous form can be additionally incorporated into the intimate dry mixture).

The starting compounds for preparing multimetal oxide active compositions I can be intimately mixed in dry or in wet form. When they are mixed in dry form, the starting compounds are advantageously used as finely divided powders and subjected to calcining after mixing and optional compaction. However, preference is given to intimate mixing in wet form. Customarily, the starting compounds are mixed with each other in the form of an aqueous solution and/or suspension. Particularly intimate dry mixtures are obtained in the mixing process described when the starting materials are exclusively sources of the elemental constituents in dissolved form. The solvent used is preferably water. Subsequently, the aqueous composition obtained is dried, and the drying process is preferably effected by spray-drying the aqueous mixture at exit temperatures from the spray tower of from 100 to 150° C.

Typically, the multimetal oxide active compositions of the general formula I are used in the fixed catalyst bed not in powder form, but rather shaped into certain catalyst geometries, and the shaping may be effected before or after the final calcination. For example, unsupported catalysts can be prepared from the powder form of the active composition or its uncalcined and/or partially calcined precursor composition by compacting to the desired catalyst geometry (for example by tableting or extruding), if appropriate with the addition of assistants, for example graphite or stearic acid as lubricants and/or shaping assistants and reinforcing agents such as microfibers of glass, asbestos, silicon carbide or potassium titanate. Examples of suitable unsupported catalyst geometries are solid cylinders or hollow cylinders having an external diameter and a length of from 2 to 10 mm. In the case of the hollow cylinder, a wall thickness of from 1 to 3 mm is advantageous. It will be appreciated that the unsupported catalyst can also have spherical geometry, and the spherical diameter may be from 2 to 10 mm.

A particularly advantageous hollow cylinder geometry is 5 mm×3 mm×2 mm (external diameter x length x internal diameter), in particular in the case of unsupported catalysts.

It will be appreciated that the pulverulent active composition or its pulverulent precursor composition which is yet to be calcined and/or partially calcined may also be shaped by applying to preshaped inert catalyst supports. The coating of the support bodies to produce the coated catalysts is generally performed in a suitable rotatable vessel, as disclosed, for example, by DE-A 29 09 671, EP-A 293 859 or EP-A 714 700. To coat the support bodies, the powder composition to be applied is appropriately moistened and dried again after application, for example by means of hot air. The coating thickness of the powder composition applied to the support body is advantageously selected within the range from 10 to 1000 μm, preferably within the range from 50 to 500 μm and more preferably within the range from 150 to 250 μm. Alternatively, the powder composition to be applied may also be applied to the support bodies directly from a suspension or solution thereof (for example in water).

Useful support materials are customary porous or nonporous aluminum oxides, silicon dioxide, thorium dioxide, zirconium dioxide, silicon carbide or silicates such as magnesium silicate or aluminum silicate. They generally behave substantially inertly with regard to the target reaction on which the process according to the invention in the first reaction stage is based. The .support bodies may have a regular or irregular shape, although preference is given to regularly shaped support bodies having distinct surface roughness, for example spheres or hollow cylinders. It is suitable to use substantially nonporous, surface-roughened spherical supports made of steatite (e.g. Steatite C220 from CeramTec) whose diameter is from 1 to 8 mm, preferably from 4 to 5 mm. However, suitable support bodies also include cylinders whose length is from 2 to 10 mm (e.g. 8 mm) and whose external diameter is from 4 to 10 mm (e.g. 6 mm). In the case of rings which are suitable in accordance with the invention as support bodies, the wall thickness is also typically from 1 to 4 mm. Annular support bodies to be used with preference in accordance with the invention have a length of from 2 to 6 mm, an external diameter of from 4 to 8 mm and a wall thickness of from 1 to 2 mm. Also suitable as support bodies in accordance with the invention are in particular rings of the geometry 7 mm×3 mm×4 mm or 5 mm×3 mm×2 mm (external diameter×length×internal diameter). It will be appreciated that the fineness of the catalytically active oxide compositions to be applied to the surface of the support body is adapted to the desired coating thickness (cf. EP-A 714 700).

Multimetal oxide active compositions which are particularly suitable for the catalysts of the fixed catalyst bed of a propylene partial oxidation to acrolein in the context of the present invention are also compositions of the general formula II

[Y¹_{a′}Y²_{b′}O_{x′}]ₚ[Y³_{c′}Y⁴_{d′}Y⁵_{e′}Y⁶_{f}Y⁷_{g′}Y²_{h′}O_{y′}]_{q} (II)

in which the variables are each defined as follows:
Y¹=only bismuth or bismuth and at least one of the elements tellurium, antimony, tin and copper,
Y²=molybdenum, or tungsten, or molybdenum and tungsten,
Y³=an alkali metal, thallium and/or samarium,
Y⁴=an alkaline earth metal, nickel, cobalt, copper, manganese, zinc, tin, cadmium and/or mercury,
Y⁵=iron or iron and at least one of the elements chromium and cerium,
Y⁶=phosphorus, arsenic, boron and/or antimony,
Y⁷=a rare earth metal, titanium, zirconium, niobium, tantalum, rhenium, ruthenium, rhodium, silver, gold, aluminum, gallium, indium, silicon, germanium, lead, thorium and/or uranium,
a′=from 0.01 to 8,
b′=from 0.1 to 30,
c′=from 0 to 4,
d′=from 0 to 20,
e′=from >0 to 20,
f′=from 0 to 6,
g′=from 0 to 15,
h′=from 8 to 16,
x′, y′=numbers which are determined by the valency and frequency of the elements in II other than oxygen and
p,q=numbers whose p/q ratio is from 0.1 to 10,
   comprising three-dimensional regions of the chemical composition Y¹_{a′}Y²_{b′}O_{x′} which are delimited from their local environment as a consequence of their different composition from their local environment, and whose maximum diameter (longest direct line passing through the center of the region and connecting two points on the surface (interface) of the region) is from 1 nm to 100 μm, frequently from 10 nm to 500 nm or from 1 μm to 50 or 25 μm.

Particularly advantageous multimetal oxide compositions II are those in which Y¹ is only bismuth.

Among these, preference is given in turn to those of the general formula III

[Bi_{a′}Z²_{b′}O_{x′}]_{p′} [Z²₁₂Z³_{c′}Z³_{c′}Z⁴_{d′}Fe_{e′}Z⁵_{f}Z⁶_{g′}Z⁷_{h′}O_{y′}]_{1′} (III)

in which the variants are each defined as follows:
Z²=molybdenum, or tungsten, or molybdenum and tungsten,
Z³=nickel and/or cobalt,
Z⁴=thallium, an alkali metal and/or an alkaline earth metal,
Z⁵=phosphorus, arsenic, boron, antimony, tin, cerium and/or lead,
Z⁶=silicon, aluminum, titanium and/or zirconium,
Z⁷=copper, silver and/or gold,
a″=from 0.1 to 1,
b″=from 0.2 to 2,
c″=from 3 to 10,
d″=from 0.02 to 2,
e″=from 0.01 to 5, preferably from 0.1 to 3,
f″=from 0 to 5,
g″=from 0 to 10,
h″=from 0 to 1,
x″,y″=numbers which are determined by the valency and frequency of the elements in III other than oxygen,
p″,q″=numbers whose p″/q″ ratio is from 0.1 to 5, preferably from 0.5 to 2,
and very particular preference is given to those compositions III in which
Z²_{b″=(tungsten)}_{b″} and Z²₁₂=(molybdenum)₁₂.

It is also advantageous when at least 25 mol % (preferably at least 50 mol % and more preferably at least 100 mol %) of the total proportion of [Y¹_{a′}Y²_{b′}O_{x′}]ₚ ([Bi_{a′}Z²_{b′}O_{x′}]_{p′}) of the multimetal oxide compositions II (multimetal oxide compositions III) suitable in accordance with the invention in the multimetal oxide compositions II (multimetal oxide compositions III) suitable in accordance with the invention are in the form of three-dimensional regions of the chemical composition Y¹_{a′}Y²_{b′}O_{x′} [Bi_{a′}Z²_{b′}O_{x′}] which are delimited from their local environment as a consequence of their different chemical composition from their local environment, and whose maximum diameter is in the range from 1 nm to 100 μm.

With regard to the shaping, the statements made for the multimetal oxide I catalysts apply to multimetal oxide II catalysts.

The preparation of multimetal oxide active compositions II is described, for example, in EP-A 575 897 and also in DE-A 198 55 913, DE-A 103 44 149 and DE-A 103 44 264.

Suitable active compositions for catalysts of a fixed catalyst bed suitable for the partial oxidation of acrolein to acrylic acid in the context of the present invention are the multimetal oxides known for this reaction type which comprise the elements Mo and V.

Such multimetal oxide active compositions comprising Mo and V can be taken, for example, from U.S. Pat. No. 3,775,474, U.S. Pat. No. 3,954,855, U.S. Pat. No. 3,893,951, and U.S. Pat. No. 4,339,355, or EP-A 614 872 or EP-A 1 041 062, or WO 03/055835, or WO 03/057653.

Especially suitable are also the multimetal oxide active compositions of DE-A 103 25 487 and also of DE-A 103 25 488.

Also particularly suitable as active compositions for the fixed bed catalysts for the partial oxidation of acrolein to acrylic acid in the context of the present invention are the multimetal oxide compositions of EP-A 427 508, DE-A 29 09 671, DE-C 31 51 805, DE-B 26 26 887, DE-A 43 02 991, EP-A 700 893, EP-A 714 700 and DE-A 197 36 105. Particular preference is given in this context to the exemplary embodiments of EP-A 714 700 and of DE-A 197 36 105.

A multitude of these multimetal oxide active compositions comprising the elements Mo and V can be encompassed by the general formula IV

Mo₁₂V_{B}X¹_{b}X²_{c}X³_{d}X⁴ₑX⁵_{f}X⁶_{g}Oₙ (IV)

in which the variables are each defined as follows:
X¹=W, Nb, Ta, Cr and/or Ce,
X²=Cu, Ni, Co, Fe, Mn and/or Zn,
X³=Sb and/or Bi,
X⁴=one or more alkali metals,
X⁵=one or more alkaline earth metals,
X⁶=Si, Al, Ti and/or Zr,
a=from 1 to 6,
b=from 0.2 to 4,
c=from 0.5 to 18,
d=from 0 to 40,
e=from 0 to 2,
f=from 0 to 4,
g=from 0 to 40 and
n=a number which is determined by the valency and frequency of the elements in IV other than oxygen.

Preferred embodiments among the active multimetal oxides IV in the context of the present invention are those which are encompassed by the following definitions of the variables of the general formula IV:
X¹=W, Nb and/or Cr,
X²=Cu, Ni, Co and/or Fe,
X³=Sb,
X⁴=Na and/or K,
X⁵=Ca, Sr and/or Ba,
X⁶=Si, Al and/or Ti,
a=from 1.5 to 5,
b=from 0.5 to 2,
c=from 0.5 to 3,
d=from 0 to 2,
e=from 0 to 0.2,
f=from 0 to 1 and
n=a number which is determined by the valency and frequency of the elements in IV other than oxygen.

However, multimetal oxides IV which are very particularly preferred in the context of the present invention are those of the general formula V

Mo₁₂V_{a′}Y¹_{b′}Y²_{c′}Y⁵_{f}Y⁶_{g′}O_{n′} (V)

where
Y¹=W and/or Nb,
Y²=Cu and/or Ni,
Y⁵=Ca and/or Sr,
Y⁶=Si and/or Al,
a′=from 2 to 4,
b′=from 1 to 1.5,
c′=from 1 to 3,
f′=from 0 to 0.5
g′=from 0 to 8 and
n′=a number which is determined by the valency and frequency of the elements in V other than oxygen.

Multimetal oxide active compositions (IV) are obtainable in a manner known per se, for example disclosed in DE-A 43 35 973 or in EP-A 714 700. In particular, suitable multimetal oxide active compositions comprising Mo and V in the context of the present invention for the partial oxidation of acrolein to acrylic acid are also the multimetal oxide active compositions of DE-A 102 61 186.

In principle, such multimetal oxide active compositions comprising Mo and V, especially those of the general formula IV, can be prepared in a simple manner by obtaining a very intimate, preferably finely divided dry mixture having a composition corresponding to their stoichiometry from suitable sources of their elemental constituents and calcining it at temperatures of from 350 to 600° C. The calcination may be carried out either under inert gas or under an oxidative atmosphere, for example air (mixture of inert gas and oxygen), and also under a reducing atmosphere (for example mixtures of inert gas and reducing gases such as H₂, NH₃, CO, methane and/or acrolein or the reducing gases mentioned themselves). The calcination time can be from a few minutes to a few hours and typically decreases with temperature. Useful sources for the elemental constituents of the multimetal oxide active compositions IV include those compounds which are already oxides and/or those compounds which can be converted to oxides by heating, at least in the presence of oxygen.

The starting compounds for preparing multimetal oxide compositions IV can be intimately mixed in dry or in wet form. When they are mixed in dry form, the starting compounds are advantageously used as finely divided powder and subjected to calcining after mixing and optional compaction. However, preference is given to intimate mixing in wet form.

Customarily, the starting compounds are mixed with each other in the form of an aqueous solution and/or suspension. Particularly intimate dry mixtures are obtained in the mixing process described when the starting materials are exclusively sources of the elemental constituents in dissolved form. The solvent used is preferably water. Subsequently, the aqueous composition obtained is dried, and the drying process is preferably effected by spray-drying the aqueous mixture at exit temperatures of from 100 to 150° C.

The multimetal oxide active compositions comprising Mo and V, especially those of the general formula IV, may be used for the process according to the invention of a partial oxidation of acrolein to acrylic acid either in powder form or shaped to certain catalyst geometries, and the shaping may be effected before or after the final calcination. For example, unsupported catalysts can be prepared from the powder form of the active composition or its uncalcined precursor composition by compacting to the desired catalyst geometry (for example by tableting or extruding), if appropriate with the addition of assistants, for example graphite or stearic acid as lubricants and/or shaping assistants and reinforcing agents such as microfibers of glass, asbestos, silicon carbide or potassium titanate. Examples of suitable unsupported catalyst geometries are solid cylinders or hollow cylinders having an external diameter and a length of from 2 to 10 mm. In the case of the hollow cylinder, a wall thickness of from 1 to 3 mm is advantageous. It will be appreciated that the unsupported catalyst may also have spherical geometry, and the spherical diameter may be from 2 to 10 mm.

It will be appreciated that the pulverulent active composition or its pulverulent precursor composition which is yet to be calcined may also be shaped by applying to preshaped inert catalyst supports. The coating of the support bodies to produce the coated catalysts is generally performed in a suitable rotatable vessel, as disclosed, for example, by DE-A 29 09 671, EP-A 293 859 or by EP-A 714 700.

To coat the support bodies, the powder composition to be applied is appropriately moistened and dried again after application, for example by means of hot air. The coating thickness of the powder composition applied to the support body is advantageously selected within the range from 10 to 1000 μm, preferably within the range from 50 to 500 μm and more preferably within the range from 150 to 250 μm.

Useful support materials are customary porous or nonporous aluminum oxides, silicon dioxide, thorium dioxide, zirconium dioxide, silicon carbide or silicates such as magnesium silicate or aluminum silicate. The support bodies may have a regular or irregular shape, although preference is given to regularly shaped support bodies having distinct surface roughness, for example spheres or hollow cylinders. It is suitable to use substantially nonporous, surface-roughened, spherical supports made of steatite whose diameter is from 1 to 10 mm (e.g. 8 mm), preferably from 4 to 5 mm. However, suitable support bodies also include cylinders whose length is from 2 to 10 mm and whose external diameter is from 4 to 10 mm. In the case of rings which are suitable in accordance with the invention as support bodies, the wall thickness is also typically from 1 to 4 mm. Annular support bodies to be used with preference in accordance with the invention have a length of from 3 to 6 mm, an external diameter of from 4 to 8 mm and a wall thickness of from 1 to 2 mm. Suitable support bodies according to the invention are also in particular rings of geometry 7 mm×3 mm×4 mm (external diameter x length x internal diameter). It will be appreciated that the fineness of the catalytically active oxide compositions to be applied to the surface of the support body is adapted to the desired coating thickness (cf. EP-A 714 700). Favorable multimetal oxide active compositions comprising Mo and V and which are to be used in the context of the present invention for an acrolein partial oxidation to acrylic acid are also compositions of the general formula VI

[D]ₚ[E]_{q} (VI)

in which the variables are each defined as follows:
D=Mo₁₂Va_{a″}Z¹_{b″}Z²_{c″}Z³_{d″}Z⁴_{e″}Z⁵_{f″}Z⁶_{g″}O_{x″},
E=Z⁷₁₂Cu_{h″}H_{i″}O_{y″},
Z¹=W, Nb, Ta, Cr and/or Ce,
Z²=Cu, Ni, Co, Fe, Mn and/or Zn,
Z³=Sb and/or Bi,
Z⁴=Li, Na, K, Rb, Cs and/or H,
Z⁵=Mg, Ca, Sr and/or Ba,
Z⁶=Si, Al, Ti and/or Zr,
Z⁷=Mo, W, V, Nb and/or Ta,
a″=from 1 to 8,
b″=from 0.2 to 5,
c″=from 0 to 23,
d″=from 0 to 50,
e″=from 0 to 2,
f″=from 0 to 5,
g″=from 0 to 50,
h″=from 4 to 30,
i″=from 0 to 20 and
x″, y″=numbers which are determined by the valency and frequency of the elements in VI other than oxygen and
p, q=numbers other than zero whose p/q ratio is from 160:1 to 1:1,

and which are obtainable by separately preforming a multimetal oxide composition E

Z⁷₁₂Cu_{h″}H_{i″}O_{y″} (E)

in finely divided form (starting composition 1) and subsequently incorporating the preformed solid starting composition 1 into an aqueous solution, an aqueous suspension or into a finely divided dry mixture of sources of the elements Mo, V, Z¹, Z², Z³, Z⁴, Z⁵, Z⁶which comprises the abovementioned elements in the stoichiometry D

Mo₁₂V_{a″}Z¹_{b″}Z²_{c″}Z³_{d″}Z⁴_{e″}Z⁴_{e″}Z⁵_{f″}Z⁶_{g″} (D)

(starting composition 2) in the desired p:q ratio, drying the aqueous mixture which may result, and calcining the resulting dry precursor composition before or after drying at temperatures of from 250 to 600° C. to give the desired catalyst geometry.

Preference is given to those multimetal oxide active compositions VI in which the preformed solid starting composition 1 is incorporated into an aqueous starting composition 2 at a temperature of <70° C. A detailed description of the preparation of multimetal oxide composition III catalysts is comprised, for example, in EP-A 668 104, DE-A 197 36 105 and DE-A 195 28 646.

With regard to the shaping, the statements made for the multimetal oxide active composition VI catalysts apply to multimetal oxide active composition VI catalysts.

Further multimetal oxide active compositions comprising Mo and V which are favorable in the context described are also multielement oxide active compositions of the general formula VII

[A]ₚ[B]_{q}[C]ᵣ (VII)

in which the variables are each defined as follows:
A=Mo₁₂VₐX¹_{b}X²_{c}X³_{d}X⁴ₑX⁵_{f}X⁶_{g}Oₓ,
B=X₁⁷CuₕHᵢO_{y},
C=X₁⁸SbⱼHₖO_{z},
X¹=W, Nb, Ta, Cr and/or Ce, preferably W, Nb and/or Cr,
X²=Cu, Ni, Co, Fe, Mn and/or Zn, preferably Cu, Ni, Co and/or Fe,
X³=Sb and/or Bi, preferably Sb,
X⁴=Li, Na, K, Rb, Cs and/or H, preferably Na and/or K,
X⁵=Mg, Ca, Sr and/or Ba, preferably Ca, Sr and/or Ba,
X⁶=Si, Al, Ti and/or Zr, preferably Si, Al and/or Ti,
X⁷=Mo, W, V, Nb-and/or Ta, preferably Mo and/or W,
X⁸=Cu, Ni, Zn, Co, Fe, Cd, Mn, Mg, Ca, Sr and/or Ba, preferably Cu and/or Zn, more preferably Cu,
a=from 1 to 8, preferably from 2 to 6,
b=from 0.2 to 5, preferably from 0.5 to 2.5
c=from 0 to 23, preferably from 0 to 4,
d=from 0 to 50, preferably from 0 to 3,
e=from 0 to 2, preferably from 0 to 0.3,
f=from 0 to 5, preferably from 0 to 2,
g=from 0 to 50, preferably from 0 to 20,
h=from 0.3 to 2.5, preferably from 0.5 to 2, more preferably from 0.75 to 1.5,
i=from 0 to 2, preferably from 0 to 1,
j=from 0.1 to 50, preferably from 0.2 to 20, more preferably from 0.2 to 5,
k=from 0 to 50, preferably from 0 to 20, more preferably from 0 to 12,
x, y, z=numbers which are determined by the valency and frequency of the elements in A, B, C other than oxygen,

p, q=positive numbers
r=6b 0 or a positive number, preferably a positive number, where the p/(q+r) ratio=from 20:1 to 1:20, preferably from 5:1 to 1:14 and more preferably from 2:1 to 1:8 and, in the case that r is a positive number, the q/r ratio=from 20:1 to 1:20, preferably from 4:1to 1:4, more preferably from 2:1 to 1:2 and most preferably 1:1,

which comprise the fraction [A]ₚ in the form of three-dimensional regions (phases) A of the chemical composition

A: Mo₁₂VaX¹_{b}X²_{c}X³_{d}X⁴ₑX⁵_{f}X⁶_{g}Oₓ,

the fraction [B]q in the form of three-dimensional regions (phases) B of the chemical composition

B: X₁⁷CuₕHᵢO_{y} and

the fraction [C]ᵣ in the form of three-dimensional regions (phases) C of the chemical composition

C: X₁⁸SbⱼHₖO_{z},

where the regions A, B and, where present, C are distributed relative to each other as in a mixture of finely divided A, finely divided B and, where present, finely divided C, and where all variables are to be selected within the predefined ranges with the proviso that the molar fraction of the element Mo in the total amount of all elements in the multielement oxide active composition VII other than oxygen is from 20 mol % to 80 mol %, the molar ratio of Mo present in the catalytically active multielement oxide composition VII to V present in the catalytically active multielement oxide composition VII, MoN, is from 15:1 to 1:1, the corresponding molar Mo/Cu ratio is from 30:1 to 1:3 and the corresponding molar Mo/(total amount of W and Nb) ratio is from 80:1 to 1:4.

Multielement oxide active compositions VII preferred in the context of the present invention are those whose regions A have a composition within the following stoichiometric pattern of the general formula VIII:

Mo₁₂VₐX¹_{b}X²_{c}X⁵_{f}X⁶_{g}Oₓ (VIII)
where
X¹=W and/or Nb,
X²=Cu and/or Ni,
X⁵=Ca and/or Sr,
X⁶=Si and/or Al,
a=from 2 to 6,
b=from 1 to 2,
c=from 1 to 3,
f=from 0 to 0.75;
g=from 0 to 10, and
x=a number which is determined by the valency and frequency of the elements in (VIII) other than oxygen.

The term “phase” used in connection with the multielement oxide active compositions VIII means three-dimensional regions whose chemical composition is different to that of their environment. The phases are not necessarily x-ray-homogeneous. In general, phase A forms a continuous phase in which particles of phase B and, where present, C are dispersed.

The finely divided phases B and, where present, C advantageously consist of particles whose largest diameter, i.e. longest line passing through the center of the particles and connecting two points on the surface of the particles, is up to 300 μm, preferably from 0.1 to 200 μm, more preferably from 0.5 to 50 μm and most preferably from 1 to 30 μm. However, particles having a largest diameter of from 10 to 80 μm or from 75 to 125 μm are also suitable.

In principle, the phases A, B and, where present, C may be in amorphous and/or crystalline form in the multielement oxide active compositions VII.

The intimate dry mixtures on which the multielement oxide active compositions of the general formula VII are based and which are subsequently to be treated thermally to convert them to active compositions may be obtained, for example, as described in the documents WO 02/24327, DE-A 44 05 514, DE-A 44 40 891, DE-A 195 28 646, DE-A 197 40 493, EP-A 756 894, DE-A 198 15 280, DE-A 198 15 278, EP-A 774 297, DE-A 198 15 281, EP-A 668 104 and DE-A 197 36 105.

The basic principle of preparing intimate dry mixtures whose thermal treatment leads to multielement oxide active compositions of the general formula VII is to preform, in finely divided form, separately or combined together, at least one multielement oxide composition B (X₁⁷CUₕHᵢO_{y}) as the starting composition 1 and, if appropriate, one or more multielement oxide compositions C (X₁⁸SbⱼHₖO_{z}) as the starting composition 2, and subsequently to intimately contact, in the desired ratio (corresponding to the general formula VII), the starting compositions 1 and, if appropriate, 2 with a mixture which comprises sources of the elemental constituents of the multielement oxide composition A

Mo₁₂VₐV_{b}¹X_{c}²X_{d}³Xₑ⁴X_{f}⁵X_{g}⁶Oₓ (A)

in a composition corresponding to the stoichiometry A, and if appropriate to dry the resulting intimate mixture.

The intimate contacting of the constituents of the starting compositions 1 and, if appropriate, 2 with the mixture comprising the sources of the elemental constituents of the multimetal oxide composition A (starting composition 3) may be effected either in dry or in wet form. In the latter case, care has to be taken merely that the preformed phases (crystals) B and, if appropriate, C do not go into solution. In an aqueous medium, the latter is usually ensured at pH values which do not deviate too far from 7 and at temperatures which are not excessively high. When the intimate contacting is effected in wet form, there is normally final drying to give the intimate dry mixture to be thermally treated in accordance with the invention (for example by spray-drying). In the case of dry mixing, such a dry mass is obtained automatically. It will be appreciated that the phases B and, if appropriate, C preformed in finely divided form may also be incorporated into a plastically reshapeable mixture which comprises the sources of the elemental constituents of the multimetal oxide composition A, as recommended by DE-A 100 46 928. The intimate contacting of the constituents of the starting compositions 1 and, if appropriate, 2 with the sources of the multielement oxide composition A (starting composition 3) may of course also be effected as described in DE-A 198 15 281.

The thermal treatment to obtain the active composition and the shaping may be effected as described for the multimetal oxide active compositions IV to VI.

Quite generally, multimetal oxide active compositions IV to VII catalysts may advantageously be prepared in accordance with the teaching of DE-A 103 25 487 or DE-A 103 25 488.

The performance of a heterogeneously catalyzed partial exothermic gas phase oxidation suitable for the process according to the invention can be carried out with the catalysts described as suitable for the fixed catalyst bed in question, in the simplest manner and appropriately from an application point of view, in a tube bundle reactor which has only one temperature zone and has been charged with the fixed catalyst bed, as described, for example, in EP-A 700 893, in EP-A 700 714 or DE-A 44 31 949, or WO 03/057653, or WO 03/055835, or WO 03/059857, or WO 03/076373, or EP-A 1 695 954 (FIG. 1) and the prior art cited in these documents.

In other words, in the simplest manner, the fixed catalyst bed is disposed in the preferably uniformly charged (metal) tubes of a tube bundle reactor and a fluid heat carrier (a heating medium; generally a salt melt) is conducted through the space surrounding the reaction tubes. The fluid heat carrier (the heating medium; e.g. the salt melt) and reaction gas mixture may be conducted in simple co- or countercurrent. However, the fluid heat carrier (the heating medium; e.g. the salt melt) may also be conducted around the tube bundle in a meandering manner viewed over the reactor, so that only viewed over the entire reactor does a co- or countercurrent to the flow direction of the reaction gas mixture exist (cf. EP-A 700 893 and EP-A 1 695 954). Irrespective of the detailed flow control of the fluid heat carrier (of the heating medium; e.g. of the salt melt), the volume flow rate thereof is typically such that the temperature rise (caused by the exothermicity of the reaction) of the fluid heat carrier (of the heating medium; e.g. of the salt melt) from the inlet point into the space surrounding the reaction tubes to the outlet point from the space surrounding the reaction tubes (i.e. T^{out}-Tⁱⁿ) is from 0 to 10° C., frequently from 2 to 8° C., often from 3 to 6° C. The inlet temperature of the fluid heat carrier into the space surrounding the catalyst tubes (Tⁱⁿ) is generally from 250 to 450° C., frequently from 300 to 400° C. or from 300 to 380° C.

The reaction temperature within the reaction tubes likewise varies predominantly within the aforementioned temperature range. Over wide ranges of the catalyst tube, the reaction temperature (the temperature of the fixed catalyst bed) is ≧Tⁱⁿ.

The maximum reaction temperature along the catalyst tube, T^{max} (the temperature of the hotspot), may be up to 70° C. or more above Tⁱⁿ. The difference T^{max}-Tⁱⁿ is referred to as the hotspot expansion ΔT^{H}.

In general, it is attempted to avoid a ΔT^{H} of ≧80° C. Usually, ΔT^{H} is ≦70° C., frequently from 20 to 70° C., and ΔT^{H} is preferably small. In other words, the fixed catalyst bed, the reaction gas input mixture, the load of the fixed catalyst bed with reaction gas input mixture and the removal of the heat of reaction with the aid of the fluid heat carrier are normally configured in such a way that the aforementioned values for ΔT^{H} are attained at the desired space-time yields of target product.

Moreover, the catalyst selection for the fixed catalyst bed and its possible dilution with inert material in the fixed catalyst bed is normally effected (cf. EP-A 990 636 and EP-A 1 106 598) in such a way that, in the freshly charged fixed catalyst bed in the predefined reaction tube flowed around by heat carrier, ΔT^{H} on increase of Tⁱⁿ by +1° C. is normally ≦9° C., preferably ≦7° C., or ≦5° C., or ≦3° C.

All of the above is especially true with reference to all reactant conversions reported as possible in this document (based on a single pass of the reaction gas mixture through the fixed catalyst bed).

As the fluid heat carrier, it is found to be advantageous from an application point of view to use melts of salts such as potassium nitrate, potassium nitrite, sodium nitrite and/or sodium nitrate, or of low-melting metals such as sodium, mercury and alloys of different metals. However, ionic liquids are also usable.

Appropriately, the reaction gas input mixture is fed to the fixed catalyst bed essentially preheated to Tⁱⁿ (this measure minimizes, inter alia, radial temperature gradients within the fluid heat carrier over a cross section of the tube bundle reactor in the region of the entry of the reaction gas input mixture into the tube bundle reactor).

Especially in the case of desired elevated (e.g. ≧110 I (STP)/I·h, or ≧130 I (STP)/I·h, or ≧140 I (STP)/I619 h, or ≧150 I (STP)/I·h, or ≧160 I (STP)/I·h, but generally ≦600 I (STP)/I·h, frequently ≦350 I (STP)/I·h) loads of the fixed catalyst bed with propylene (but also quite possibly in the range of propylene loads of the fixed catalyst bed of ≧60 I (STP)/I·h and <110 I (STP)/I·h), the propylene partial oxidation process to give acrolein and/or acrylic acid is performed appropriately in a two-zone or multizone tube bundle reactor (performance in a one-zone tube bundle reactor is, however, likewise possible). A preferred variant of a two-zone tube bundle reactor usable in accordance with the invention for this purpose is disclosed by DE-C 28 30 765. However, the two-zone tube bundle reactors disclosed in DE-C 25 13 405, U.S. Pat. No. 3,147,084, DE-A 22 01 528, EP-A 383 224 and DE-A 29 03 582 are also suitable. Multizone variants are also described in EP-A 1 106 598, WO 2004/085362, WO 2004/085370 and WO 2004/085363.

In other words, in a simple manner, the fixed catalyst bed is then conducted into the preferably uniformly charged (metal) tubes of a tube bundle reactor, and two (or more) essentially spatially separate (separated from one another, for example, by means of separating walls through whose bores the reaction tubes are conducted) fluid heat carriers (generally salt melts) are conducted around the reaction tubes.

The tube section (the surrounding space segment) over which the particular heat carrier (the particular salt bath) extends represents one temperature zone.

For example, a salt bath A preferably flows around that (longitudinal) section A of the tubes (temperature zone A) in which the oxidative conversion of propylene (in single pass) proceeds until a conversion value C_{A} in the range from 15 to 85 mol % is achieved and a salt bath B preferably flows around the (longitudinal) section B of the tubes (temperature zone B) in which the subsequent oxidative conversion of propylene (in single pass) until a conversion value C_{B} of generally at least 90 mol % (preferably ≧92 mol %, or ≧94 mol %, or ≧96 mol %) is achieved (if required, temperature zones A, B may be followed by further temperature zones which are kept at individual temperatures).

Within the particular temperature zone, the particular salt bath may in principle be conducted as in the one-zone method (in particular relative to the reaction gas mixture). In other words, salt bath A is conducted into temperature zone A with the temperature T^{in,A} and out again with the temperature T^{out,A}, where T^{out,A}>T^{in,A}. In a corresponding manner, salt bath B is conducted into temperature zone B with the temperature T^{in,B} and out again with the temperature T^{out,B}, where T^{out,B}>^{Tin,B} (at this point, it should be emphasized that the terms “salt bath A” and “salt bath B” in this document always represent a fluid heat carrier A or a fluid heat carrier B, so that the particular disclosure also comprises the wording formed by replacing “salt bath A” with “a fluid heat carrier A” and “salt bath B” with “a fluid heat carrier B”).

The fluid heat carrier (the heating medium; generally a salt melt) A (B) and the reaction gas mixture may be conducted either in simple cocurrent or in simple countercurrent within temperature zone A (B). However, the fluid heat carrier (the heating medium; e.g. the salt melt) A (B) may also be conducted in a meandering manner around the tube bundle section A (B), so that only viewed over the entire tube bundle section A (B) does a cocurrent or countercurrent to the flow direction of the reaction gas mixture exist. Irrespective of the detailed flow control of the fluid heat carrier (of the heating medium; e.g. of the salt melt) A (B), the volume flow rate thereof is typically such that the temperature rise (caused by the exothermicity of the reaction) of the fluid heat carrier (of the heating medium; e.g. of the salt melt) A (B) from the inlet point into the space surrounding the tube bundle section A (B) to the outlet point from the space surrounding the tube bundle section A (B) (i.e. T^{out,A}-T^{in,A}(T^{out,B}-T^{in,B})) is from 0 to 10° C., frequently from 2 to 8° C., often from 3 to 6° C.

The maximum reaction temperature (the temperature of the particular hotspot) along the catalyst tube section A, T^{maxA} (of the catalyst tube section B, T^{maxB}), may be up to 70° C. or more above T^{in,A}(T^{in,B}). The difference T^{maxA}-T^{in,A}(T^{maxB}-T^{in,B}) is referred to as the hotspot expansion ΔT^{H,A}(ΔT^{H,B}).

According to the teaching of the prior art, the general process conditions in the freshly charged fixed catalyst bed are generally selected such that T^{maxA}-T^{maxB} is ≧0° C. and ≦80° C. (cf., for example, WO 2004/085362, WO 2004/085370, WO 2004/085363 and in European application 06 100 535).

At relatively low reactant loads of the fresh fixed catalyst bed, this is frequently advantageously satisfied when T^{in,B}-T^{in,A} is <0° C., while, with increasing reactant load of the fresh fixed catalyst bed, the above condition is frequently advantageously satisfied when T^{in,B}-T^{in,A} is ≧0° C.

Moreover, the catalyst selection for the fixed catalyst bed and its possible dilution with inert material in the fixed catalyst bed is normally effected (cf. EP-A 990 636 and EP-A 1 106 598) such that, in the freshly charged fixed catalyst bed in the predefined reaction tube section flowed around by the heat carrier, ΔT^{H,A}(ΔT^{H,B}) on increase of T^{in,A}(T^{in,B}) by +1° C., is normally ≦9° C., preferably ≦8° C., more preferably ≦7° C., or ≦5° C., or ≦3° C.

The entrance temperature of the particular fluid heat carrier into the space surrounding the particular catalyst tube section (T^{in,A} or T^{in,B}) is generally from 250 to 450° C., frequently from 300 to 400° C. or from 300 to 380° C.

In order to counteract the: deactivation of the fixed catalyst bed in long-term operation, the procedure will preferably be as described in European application 06 100 535 and DE-A 10 2004 025 445. Quite generally, in long-term operation, C_{A}=from 15 to 85 mol % and C_{B}≧90 mol % will generally be maintained.

As the fluid heat carrier in a multizone operating mode too, it has been found to be appropriate from an application point of view to use melts of salts such as potassium nitrate, potassium nitrite, sodium nitrite and/or sodium nitrate, or of low-melting metals such as sodium, mercury and alloys of different metals. However, ionic liquids are also usable. Appropriately from an application point of view, in a multizone operating mode, the same type of heat carrier will be used in each case for the different temperature zones.

In principle, in an inventive two-zone propylene partial oxidation to acrolein and/or acrylic acid in operating state II, both T_{II}^{in,A}>T_{I}^{in,A} and T_{II}^{in,B}>T_{I}^{in,B} may be satisfied. In general, at the transition from an operating state I with a propylene load of the fixed catalyst bed L^{I} to an operating state II with a propylene load of the fixed catalyst bed L^{II} >L^{I} in the two-zone tube bundle reactor described, appropriately from an application point of view, both T_{I}^{in,A} will be increased to T_{II}^{in,A} and T_{I}^{in,B} to T_{II}^{in,B} in advance of the increase in the propylene load of the fixed catalyst bed (appropriately from an application point of view, the procedure will be such that T_{II}^{in,A}-T_{I}^{in,A} will be selected to be about the same as T_{II}^{in,B}-T_{I}^{in,B}).

However, the procedure may also be, at the transition from operating state I to operating state II, only to increase T_{I}^{in,A} to T_{II}^{in,A} and to essentially retain T_{I}^{in,B}(=T_{II}^{in,B}), or only to increase T_{I}^{in,B} to T_{II}^{in,B} and to essentially retain T_{I}^{in,A}(=T_{II}^{in,A}). However, advantageously in accordance with the invention, the procedure in all three possible cases is such that (especially over the fresh fixed catalyst bed), both in operating state I and in operating state II, T^{maxA}-T^{maxB} is ≧0° C. and ≦80° C. In other words, both in operating state I and in operating state II, T^{maxA}-T^{maxB} may advantageously (and independently) be ≧1° C. and ≦70° C., or ≧2° C. and ≦60° C., or ≧3° C. and ≦50° C., or ≧4° C. and ≦40° C., or ≧5° C. and ≦30° C., or ≧5° C. and ≦25° C., or ≧5° C. and ≦20° C. and ≦15° C., or ≧0° C. and ≦5° C. It is favorable in accordance with the invention that, when T^{maxA}-T^{maxB} in operating state I is within a particular aforementioned temperature difference interval, T^{maxA}-T^{maxB} in operating state II is also within this temperature difference interval.

In principle, in operating state I, T^{maxA}-T^{maxB}(T_{I}^{maxA}-T_{I}^{maxB}) may also be ≧0° C. and ≦80° C., and, in operating state II (T_{II}^{maxA}-T_{II}^{maxB})<0° C. (for example down to −20° C., or down to −10° C., or down to −5° C.).

When, at the transition from operating state I to operating state II, both T_{I}^{in,A} and T_{I}^{in,B} are increased (to T_{II}^{in,A}-T_{II}^{in,B}) respectively), these two temperature increases can be undertaken either simultaneously (synchronously) or offset in time. In the case of an increase offset in time, it is advantageous from an application point of view to increase first T_{I}^{in,A} to T_{II}^{in,A} and then T_{I}^{in,B} to T_{II}^{in,B} (in principle, the procedure may also be reversed).

Quite generally, the catalysts and other process conditions to be used in the context of the inventive procedure should, appropriately from an application point of view, be selected such that the selectivity of target product formation, based on single pass of the reaction gas mixture through the fixed catalyst bed, is ≧80 mol %, or ≧90 mol %, in may cases even ≧92 mol %, or ≧94 mol %, or ≧96 mol %.

Moreover, the radial temperature gradient of the particular heat carrier within a temperature zone of a tube bundle reactor to be operated in accordance with the invention is kept very low. In general, this radial temperature gradient in practical application is from 0.01 to 5° C., frequently from 0.1 to 2° C.

Typically, the catalyst tubes of tube bundle reactors to be used in accordance with the invention are manufactured from ferritic steel and typically have a wall thickness of from 1 to 3 mm. Their internal diameter is generally from 20 to 30 mm, frequently from 21 to 26 mm. Their length, especially in the case of an inventive partial oxidation of propylene to acrolein or of acrolein to acrylic acid, is from 3 to 4, frequently 3.5 m. Appropriately from an application point of view, the number of catalyst tubes accommodated in the tube bundle vessel (especially in the case of the two aforementioned partial oxidations) is at least 5000, preferably at least 10 000. Frequently, the number of catalyst tubes accommodated in the reaction vessel is from 15 000 to 30 000 or to 40 000. Tube bundle reactors having a number of catalyst tubes above 50 000 are usually the exception. Within the vessel, the catalyst tubes are normally arranged in homogeneous distribution (preferably 6 equidistant neighboring tubes per catalyst tube), the distribution appropriately being selected such that the separation of the central internal axes of mutually adjacent catalyst tubes (the so-called catalyst tube pitch) is from 35 to 45 mm (cf. EP-A 468 290).

Especially in the case of desired elevated (e.g. ≧100 I (STP)/I·h, or ≧120 I (STP)/I·h, or ≧130 I (STP)/I·h, or ≧140 I (STP)/I·h, or ≧150 I (STP)/I·h, or ≧160 I (STP)/I·h, but generally ≦175 I (STP)/I·h, or ≦200 I (STP)/I·h, or ≦300 I (STP)/I·h, or normally ≦600 I (STP)/I·h) loads of the fixed catalyst bed with acrolein (but also quite possibly in the range of acrolein loads of the fixed catalyst bed of ≧50 I (STP)/I·h and ≦100 I (STP)/I·h), the acrolein partial oxidation to acrylic acid is performed in a manner corresponding to the propylene partial oxidation appropriately in a two-zone or multizone tube bundle reactor (performance in a one-zone tube bundle reactor is, however, likewise possible). A preferred variant of a two-zone tube bundle reactor usable in accordance with the invention for this purpose is disclosed by DE-C 28 30 765. However, the two-zone tube bundle reactors disclosed in DE-C 25 13 405, U.S. Pat. No. 3,147,084, DE-A 22 01 528, EP-A 383 224 and DE-A 29 03 582 are also suitable. Multizone variants are also described in EP-A 1 106 598, WO 2004/085362, WO 2004/085370 and WO 2004/085363.

In other words, in a simple manner, the fixed catalyst bed is then conducted into the preferably uniformly charged (metal) tubes of a tube bundle reactor, and two (or more) essentially spatially separate (separated from one another, for example, by means of separating walls through whose bores the reaction tubes are conducted) fluid heat carriers (generally salt melts) are conducted around the reaction tubes. The tube section (the surrounding space segment) over which the particular heat carrier (the particular salt: bath) extends represents one temperature zone.

For example, a salt bath C preferably flows around that (longitudinal) section C of the tubes (temperature zone C) in which the oxidative conversion of acrolein (in single pass) proceeds until a conversion value C_{C} in the range from 15 to 89 mol % is achieved and a salt bath D preferably flows around the (longitudinal) section D of the tubes (temperature zone D) in which the subsequent oxidative conversion of acrolein (in single pass) until a conversion value C_{D} of generally at least 90 mol % (preferably ≧92 mol %, or ≧94 mol %, or ≧96 mol %, or ≧98 mol % and frequently even ≧99 mol % and more) is achieved (if required, temperature zones C, D may be followed by further temperature zones which are kept at individual temperatures).

Within the particular temperature zone, the particular salt bath (the particular fluid heat carrier) may be conducted as in the one-zone method (in particular relative to the reaction gas mixture). In other words, salt bath C is conducted into temperature zone C with the temperature T^{in,C} and out again with the temperature T^{out,C}, where T^{out,C}>T^{in,C}. In a corresponding manner, salt bath D is conducted into temperature zone D with the temperature T^{in,D} and out again with the temperature T^{outD}, where T^{out,D}>T^{in,D} (at this point too, it should be emphasized again that the terms “salt bath C” and “salt bath D” in this document always represent a fluid heat carrier C or a fluid heat carrier D, so that the particular disclosure also comprises the wording formed by replacing “salt bath C” with “a fluid heat carrier C” and “salt bath D” with “a fluid heat carrier D”). The fluid heat carrier (the heating medium; generally a salt melt) C (D) and the reaction gas mixture may be conducted either in simple cocurrent or in simple countercurrent within temperature zone C (D). However, the fluid heat carrier (the heating medium; e.g. the salt melt) C (D) may also be conducted in a meandering manner around the tube bundle section C (D), so that only viewed over the entire tube bundle section C (D) does a cocurrent or countercurrent to the flow direction of the reaction gas mixture exist. Irrespective of the detailed flow control of the fluid heat carrier (of the heating medium; e.g. of the salt melt) C (D), the volume flow rate thereof is typically such that the temperature rise (caused by the exothermicity of the reaction) of the fluid heat carrier (of the heating medium; e.g. of the salt melt) C (D) from the inlet point into the space surrounding the tube bundle section C (D) to the outlet point from the space surrounding the tube bundle section C (D) (i.e. T^{out,c}-T^{in,c}(T^{out,D}-T^{in,D})) is from 0 to 10° C., frequently from 2 to 8° C., often from 3 to 6° C.

The maximum reaction temperature (the temperature of the particular hotspot) along the catalyst tube section C, T^{maxC} (of the catalyst tube section D, T^{maxD}), may be up to 70° C. or more above T^{in,C} (T^{in,D}).

The difference T^{maxC}-T^{in,c}(T^{maxD}-T^{in,D}) is referred to as the hotspot expansion ΔT^{H,C} (ΔT^{H,D}).

According to the teaching of the prior art, the general process conditions in the freshly charged fixed catalyst bed are generally selected such that T^{maxC}-T^{maxD} is >0° C. and ≦80° C. (cf., for example, WO 2004/085362, WO 2004/085370, WO 2004/085363 and in European application 06 100 535).

At relatively low reactant loads of the fresh fixed catalyst bed, this is frequently advantageously satisfied when T^{in,D}-T^{in,C} is <0° C., while, with increasing reactant load of the fresh fixed catalyst bed, the above condition is frequently advantageously satisfied when T^{in,D}-T^{in,C} is ≧0° C.

Moreover, the catalyst selection for the fixed catalyst bed and its possible dilution with inert material in the fixed catalyst bed is normally effected (cf. EP-A 990 636 and EP-A 1 106 598) such that, in the freshly charged fixed catalyst bed in the predefined reaction tube section flowed around by the heat carrier, ΔT^{H,C} (ΔT^{H,D}) on increase of T^{in,C}(T^{in,D}) by +1° C., is normally ≦9° C., preferably ≦8° C., more preferably ≦7° C., or ≦5° C., or ≦3° C.

The entrance temperature of the particular fluid heat carrier into the space surrounding the particular catalyst tube section (T^{in,C} or T^{in,D}) is generally from 230 to 340° C., frequently from 250 to 320° C. or from 260 to 300° C.

In order to counteract the deactivation of the fixed catalyst bed in long-term operation, the procedure will preferably be as described in European application 06 100 535 and DE-A 10 2004 025 445. Quite generally, in long-term operation, C_{C}=from 15 to 85 mol % and C_{D}≧90 mol % will generally be maintained. With regard to the useful fluid heat carriers, the same applies as was stated for the multizone propylene partial oxidation.

In principle, in an inventive two-zone acrolein partial oxidation to acrylic acid in operating state II, both T_{II}^{in,C}>T_{I}^{in,C} and T_{II}^{in,D}>T_{I}^{in,D} may be satisfied. In general, at the transition from an operating state I with a acrolein load of the fixed catalyst bed L^{I} to an operating state II with a acrolein load of the fixed catalyst bed L^{II}>L^{I} in the two-zone tube bundle reactor described, appropriately from an application point of view, both T_{I}^{in,C} will be increased to T_{II}^{in,C} and T_{I}^{in,D} to T_{II}^{in,D} in advance of the increase in the acrolein load of the fixed catalyst bed (appropriately from an application point of view, the procedure will be such that T_{II}^{in,C}-T_{I}^{in,C} will be selected to be about the same as T_{II}^{in,D}-T_{I}^{in,D}).

However, the procedure may also be, at the transition from operating state I to operating state II, only to increase T_{I}^{in,C} to T_{II}^{in,C} and to essentially retain T_{I}^{in,D}(=T_{II}^{in,D}), or only to increase T_{I}^{in,D} to T_{II}^{in,D} and to essentially retain T_{I}^{in,C}(=T_{II}^{in,C}). However, advantageously in accordance with the invention, the procedure in all three possible cases is such that (especially over the fresh fixed catalyst bed), both in operating state I and in operating state II, T^{maxC}-T^{maxD} is ≧0° C. and ≦80° C. In other words, both in operating state I and in operating state II, T^{maxC}-T^{maxD} may advantageously (and independently) be ≧1° C. and ≦70° C., or ≧2° C. and ≦60° C., or ≧3° C. and ≦50° C., or ≧4° C. and ≦40° C., or ≧5° C. and ≦30° C., or ≧5° C. and ≦25° C., or ≧5° C. and ≦20° C. and ≦15° C., or ≧0° C. and ≦5° C.

It is favorable in accordance with the invention that, when T^{maxC}-T^{maxD} in operating state I is within a particular aforementioned temperature difference interval, T^{maxC}-T^{maxD} in operating state II is also within this temperature difference interval.

In principle, in operating state I, T^{maxC}-T^{maxD}(T_{I}^{maxC}-T_{I}^{maxD}) may also be ≧0° C. and ≦80° C., and, in operating state II (T_{II}^{maxC}-T_{II}^{maxD}) <0° C. (for example down to −20° C., or down to −10° C., or down to −5° C.).

When, at the transition from operating state I to operating state II, both T_{I}^{in,C} and T_{I}^{in,D} are increased (to T_{II}^{in,C} and T_{II}^{in,D} respectively), these two temperature increases can be undertaken either simultaneously (synchronously) or offset in time. In the case of an increase offset in time, it is advantageous from an application point of view to increase first T_{I}^{in,C} to T_{II}^{in,C} and then T_{I}^{in,D} to T_{II}^{in,D} (in principle, the procedure may also be reversed).

Whether an increase from L^{I} to L^{II} is realized in the process according to the invention by an increase in the load of the fixed catalyst bed with reaction gas input mixture with essentially constant reaction gas input mixture composition and/or by an increase in the proportion of the organic starting compound in the reaction gas input mixture also depends upon the design of the separating column comprising separating internals, into which the product gas mixture is conducted in order to remove the target product. In other words, the increase of L^{I} to L^{II} will, preferably from an application point of view, be undertaken substantially such that the load of the separating column with product gas mixture remains within the range in which the separating action of the separating column is optimal. In general, this is the case when L^{I} is increased primarily by increasing the reactant proportion in the reaction gas input mixture.

Quite generally, it is desired appropriately from an application point of view in the performance of the process according to the invention to configure the compositions of the reaction gas mixture both in operating state I and in operating state II, and also in all states in the transition from operating state I to operating state II, in such a way that they are outside the explosive range, as recommended in WO 2004/007405.

Reaction gas input mixtures suitable in accordance with the invention for a propylene partial oxidation to acrolein and/or acrylic acid are, for example, those which comprise
from 5 to 15 (preferably from 7 to 11) % by volume of propylene,
from 4 to 20 (preferably from 6 to 12) % by volume of water,
from ≧0 to 10 (preferably from ≧0 to 5) % by volume of constituents other than propylene, water, molecular oxygen and molecular nitrogen,
sufficient molecular oxygen that the molar ratio of molecular oxygen present to propylene present is from 1.5 to 2.5 (preferably from 1.6 to 2.2), and as the remainder up to 100% by volume of the total amount, molecular nitrogen.

Especially at high propylene loads of the fixed catalyst bed, the additional use of inert diluent gases with high specific heat is advisable.

For example, a reaction gas input mixture for a heterogeneously catalyzed partial propylene oxidation to acrolein and/or acrylic acid may comprise up to 70% by volume of propane.

Such propylene partial oxidation reaction gas input mixtures may comprise, for example,
from 5 to 11% by volume of propylene,
from 2 to 12% by volume of water,
from >0 to 10% by volume of propane,
from ≧0 to 5% by volume of constituents other than propylene, propane, water, molecular oxygen and molecular nitrogen,
sufficient molecular oxygen that the molar ratio of molar oxygen present to propylene present is from 1 to 3, and, as the remainder up to 100% by volume of the total amount, molecular nitrogen;
   or
from 5 to 9% by volume of propylene,
from 8 to 18% by volume of molecular oxygen,
from 6 to 35% by volume of propane, and
from 32 to 72% by volume of molecular nitrogen,
   or
from 4 to 25% by volume of propylene,
from 6 to 70% by volume of propane,
from 5 to 60% by volume of H₂O,
from 8 to 65% by volume of O₂, and
from 0.3 to 20% by volume of H₂.

For an inventive heterogeneously catalyzed acrolein partial oxidation to acrylic acid, useful reaction gas input mixtures include, for example, those which comprise:
from 4.5 to 8% by volume of acrolein,
from 2.25 to 9% by volume of molecular oxygen,
from 0 to 35% by volume of propane,
from 32 to 72% by volume of molecular nitrogen, and
from 5 to 30% by volume of steam;
   or
from 3 to 25% by volume of acrolein,
from 5 to 65% by volume of molecular oxygen,
from 6 to 70% by volume of propane,
from 0 to 20% by volume of molecular nitrogen, and
from 8 to 65% by volume of steam.

Finally, it should be emphasized that, at the transition from an operating state II to an operating state I, appropriately from an application point of view, T_{II}ⁱⁿ is first reduced to T_{I}ⁱⁿ and then L^{II} is lowered to L^{I}. Advantageously, the aforementioned reductions are performed in the same manner as the inventive increases, as a sequence of small reduction steps.

In principle, the reduction of L^{II} to L^{I} can also be effected essentially simultaneously with the reduction of T_{II}ⁱⁿ to T_{I}ⁱⁿ (but in this case too preferably as a succession of small steps). The load reductions can be performed in a manner corresponding to that described in this document for the load increases, but with the difference that the parameter which is increased in the load increase is reduced in a corresponding manner in a load reduction.

In a two-stage exothermic heterogeneously catalyzed partial gas phase oxidation, at the transition from an operating state II to an operating state I, appropriately from an application point of view, T_{II}^{in,1} will first be reduced to T_{I}^{in,1} and then T_{II}^{in,2} to T_{I}^{in,2}. In a two-zone operating mode, at the transition from an operating state II to an operating state I, that heat carrier entrance temperature which had been increased first in the reverse change of operating state will normally be reduced last.

### EXAMPLE

Process for a heterogeneously catalyzed partial gas phase oxidation of propylene to acrolein in a one-zone multiple catalyst tube fixed bed reactor

Description of the general process conditions in operating state I

Heat exchange medium used: salt melt consisting of 60% by weight of potassium nitrate and 40% by weight of potassium nitrite.

Material of the catalyst tubes: ferritic steel.

Dimensions of the catalyst tubes:
length 3200 mm;
internal diameter 25 mm;
external diameter 30 mm
(wall thickness: 2.5 mm).

Number of catalyst tubes in the tube bundle: 25 500.

Reactor:
Cylindrical vessel of diameter 6800 mm; tube bundle arranged in an annular manner with a free central space.
Diameter of the central free space: 1000 mm. Separation of the outermost catalyst tubes from the vessel wall: 150 mm. Homogeneous catalyst tube distribution in the tube bundle (6 equidistant neighboring tubes per catalyst tube).
Catalyst tube pitch: 38 mm.
The catalyst tubes were secured with sealing by their ends in catalyst tube plates of thickness 125 mm, and opened at their orifices into a hood connected to the vessel at the upper or lower end in each case.
Feeding of the heat exchange medium to the tube bundle:
The tube bundle was divided by three deflecting plates (thickness in each case 10 mm) mounted in succession between the catalyst tube plates in the longitudinal direction thereof into 4 equidistant (in each case 730 mm) longitudinal sections (zones).

The lowermost and the uppermost deflecting plate had annular geometry, the internal diameter of the ring being 1000 mm and the external diameter of the ring extending up to the vessel wall with sealing. The catalyst tubes were not secured with sealing to the deflecting plates. Instead, a gap having a gap width of <0.5 mm was left such that the crossflow rate of the salt melt was substantially constant within one zone.

The middle deflecting plate was circular and extended up to the outermost catalyst tubes of the tube bundle.

The circulation of the salt melt was accomplished by two salt pumps of which each supplied one half of the tube bundle length.

The pumps injected the salt melt into an annular channel mounted at the bottom around the reactor jacket, which distributed the salt melt over the vessel circumference. The salt melt reached the tube bundle in the lowermost longitudinal section through windows present in the reactor jacket. The salt melt then flowed, following the requirements of the deflecting plates, in the sequence
from the outside inward,
from the inside outward,
from the outside inward,
from the inside outward,
   in an essentially meandering manner viewed over the vessel, from the bottom upward. The salt melt collected through windows mounted around the vessel circumference in the uppermost longitudinal section in an annular channel mounted around the reactor jacket, and was, after cooling to the original entrance temperature, injected back into the lower annular channel by the pumps.

Composition of the reaction gas input mixture (mixture of air, polymer-grade propylene and cycle gas):
5.4% by volume of propylene,
10.5% by volume of oxygen,
1.2% by volume of COₓ,
81.3% by volume of N₂,
1.6% by volume of H₂O.

Reactor charge: salt melt and reaction gas mixture were conducted in countercurrent viewed over the reactor. The salt melt entered at the bottom, the reaction gas mixture at the top. The entrance temperature of the salt melt was 337° C. The exit temperature of the salt melt was 339° C. The pump output was 6200 m³ of salt melt/h. The reaction gas input mixture was fed to the reactor with a temperature of 170° C.

Loading with reaction gas input mixture: 68 845 M³ (STP)/h.

Propylene load of the catalyst charge: 110 I(STP)/I·h.

Catalyst tube charge (from the top downward): Zone A: 50 cm
Preliminary bed of steatite rings of geometry 7 mm×7 mm×4 mm (external diameter×length×internal diameter).
Zone B: 100 cm Catalyst charge of a homogeneous mixture of 30% by weight of steatite rings of geometry 5 mm×3 mm×2 mm (external diameter x length x internal diameter) and 70% by weight of unsupported catalyst from zone C.
Zone C: 170 cm Catalyst charge of annular (5 mm×3 mm×2 mm=external diameter×length×internal diameter) unsupported catalyst according to example 1 of DE-A 10046957.

Based on single pass of the reaction gas input mixture through the fixed catalyst bed, the propylene conversion was 96.4 mol % at a selectivity of acrolein formation of 85.7 mol %.

T^{max}, determined as the mean over six thermal reaction tubes provided with multithermoelements was 398° C.

Description of the general process conditions in operating state II

As in operating state I, except that the load of the fixed catalyst bed with propylene with uniform composition of the reaction gas input mixture was 120 I(STP)/I·h, and the entrance temperature of the salt melt was 342° C. T^{max} was 403° C. Propylene conversion and selectivity of acrolein formation were as in operating state I.

The transition from operating state I to operating state II was conducted as a series of successive steps. First, the entrance temperature of the salt melt with identical propylene load was increased by 0.5° C. These operating conditions were then maintained for 1 h before the propylene load, with the entrance temperature of the salt melt kept stable, was increased sufficiently that the propylene conversion was again 96.4 mol %. The operating conditions were then retained again for 1 h before the entrance temperature of the salt melt at the new propylene load was again increased by 0.5° C., etc.

The highest T^{max} value passed through in the transition from operating state I to operating state II performed in this way was 403° C.

At no time did the propylene conversion fall below 96.4 mol %.

### COMPARATIVE EXAMPLE

When, at the transition from operating state I to operating state II with initially uniform entrance temperature of the salt melt of 337° C., the load of the fixed catalyst bed with propylene was increased from 110 I (STP)/I·h to 120 I (STP)/I·h (with uniform composition of the reaction gas input mixture), and only then was the entrance temperature of the salt melt increased from 337° C. to 342° C. (in 0.5° C. steps, each temperature increase having been maintained over 1 h before the subsequent temperature increase), the T^{max} value passed through in the transition from operating state I to operating state II performed in this way was 404° C. The propylene conversion fell temporarily below 96.4 mol %.

U.S. Provisional Patent Application Nos. 60/865929, filed Nov. 15, 2006, and 60/868631, filed Dec. 5, 2006, are incorporated into the present patent application by literature reference.

With regard to the abovementioned teachings, numerous changes and deviations from the present invention are possible.

It can therefore be assumed that the invention, within the scope of the appended claims, can be performed differently from the way described specifically herein.

1. A process for operating an exothermic heterogeneously catalyzed partial gas phase oxidation of an organic starting compound to an organic target compound in different operating states I and II, in which a reaction gas input mixture comprising the organic starting compound, molecular oxygen and at least one inert diluent gas is passed through a fixed catalyst bed disposed in the tubes of a tube bundle reactor to obtain a product gas mixture comprising the organic target compound, and the reaction temperature in the fixed catalyst bed disposed in the tubes is adjusted by conducting at least one fluid heat carrier into the space surrounding the tubes of the tube bundle reactor which comprise the fixed catalyst bed with an entrance temperature Tⁱⁿ and out of it again with an exit temperature T^{out}>Tⁱⁿ, and the fixed catalyst bed is loaded in the operating state I at an entrance temperature T_{I}ⁱⁿ with a load L^{I} of the organic starting compound and, in the operating state II, at an entrance temperature T_{II}ⁱⁿ with a load L^{II} of the organic starting compound, with the proviso that L^{II}>L^{I} and T_{II}ⁱⁿ>T_{I}ⁱⁿ, which comprises changing from operating state I to operating state II by first increasing the entrance temperature T_{I}ⁱⁿ to the value T_{II}ⁱⁿ and then increasing the loading of the fixed catalyst bed with the organic starting compound from the value L^{I} to the value L^{II}.

2. The process according to claim 1, wherein the ratio of L^{II} to L^{I} is >1 and ≦2.

3. The process according to claim 1 or 2, wherein the organic starting compound is propylene, acrolein, tert-butanol, isbutane, isobutene, isobutyraldehyde, the methyl ether of tert-butanol, methacrolein, o-xylene, p-xylene, naphthalene, butadiene, n-butane, ethylene, indane, benzene, 1-butene, 2-butene and/or ethane.

4. A process for operating a two-stage exothermic heterogenously catalyzed partial gas phase oxidation of a first organic starting compound to an organic final target compound in different operating states I and II, in which a first reaction gas input mixture comprising the first organic starting compound, molecular oxygen and at least one inert diluent gas is passed through a first fixed catalyst bed disposed in the tubes of a tube bundle reactor to obtain a first product gas mixture comprising an organic intermediate target compound, and the reaction temperature in the first fixed catalyst bed is adjusted by conducting at least one fluid heat carrier into the space surrounding the tubes of this tube bundle reactor which comprise the first fixed catalyst bed with an entrance temperature T^{1,in} and out of it again with an exit temperature T^{1,out}>T^{1,in}, and the first product gas mixture which has been cooled beforehand if appropriate and supplemented with molecular oxygen and inert gas as a secondary gas additive if appropriate is conducted as a second reaction gas input mixture comprising molecular oxygen, at least one inert diluent gas and the organic intermediate target compound as a second organic starting compound to obtain a second product gas mixture comprising the organic final target compound through a second fixed catalyst bed which is different from the first fixed catalyst bed and is disposed in the tubes of a tube bundle reactor, and the reaction temperature in the second fixed catalyst bed is adjusted by conducting at least one second fluid heat carrier with an entrance temperature T^{2,in} into the space surrounding the tubes of this tube bundle reactor which comprise the second fixed catalyst bed and out of it again with an exit temperature T^{2,out}>T^{2,in}, and, in the operating state I, the first fixed catalyst bed is loaded at an entrance temperature T_{I}^{1,in} with a load L^{1,I} of the first organic starting compound and the second fixed catalyst bed at an entrance temperature T_{I}^{2,in} with a load L^{2,I} of the second organic starting compound, and, in the operating state II, the first fixed catalyst bed is loaded at an entrance temperature T_{II}^{1,in} with a load L^{1,II} of the first organic starting compound and the second fixed catalyst bed at an entrance temperature T_{II}^{2,in} with a load L^{2,II} of the second organic starting compound, with the proviso that L^{1,II}>L^{1,I}, T_{II}^{1,in}>T_{I}^{1,in}, L^{2,II}>L^{2,I} and T_{II}^{2,in}>T_{I}^{2,in}, which comprises changing from operating state I to operating state II by first increasing the entrance temperature T_{I}^{1,in} to the value T_{II}^{1,in} and the entrance temperature T_{I}^{2,in} to the value T_{II}^{2,in}and then increasing the loading of the first fixed catalyst bed with the first organic starting compound from the value L^{1,I} to the value L^{1,II}, and, as a consequent effect of the latter measure above and of any additionally changed secondary gas addition, increasing the loading of the second fixed catalyst bed from the value L^{2,I} to the value L^{2,II}.

5. The process according to claim 4, wherein the organic starting compound is propylene, the intermediate target compound is acrolein and the final target compound is acrylic acid.
